SEQUENCE LISTING (6 sequences)

SEQ ID NO: 1 - DNA, 996 bp, Homo sapiens
ggcttagcgtggtcgcggccgaggtggcaaaaggagcatattctcaggagacggggcccctgcctgccacaccaagcattaggccaccaggaagacccccatctgcaagcaagcctagccttccagggagaaagaggcctctgcagctccttcatcatgaactggcacatgatcatctctgggcttattgtggtagtgcttaaagttgttggaatgaccttatttctactttatttcccacagatttttaacaaaagtaacgatggtttcaccaccaccaggagctatggaacagtctcacagatttttgggagcagttccccaagtcccaacggcttcattaccacaaggagctatggaacagtctgccccaaagactgggaattttatcaagcaagatgttttttcttatccacttctgaatcatcttggaatgaaagcagggacttttgcaaaggaaaaggatccacattggcaattgtcaacacgccagagaaactgtttcttcaggacataactgatgctgagaagtattttattggcttaatttaccatcgtgaagagaaaaggtggcgttggatcaacaactctgtgttcaatggcaatgttaccaatcagaatcagaatttcaactgtgcgaccattggcctaacaaagacctttgatgctgcatcatgtgacatcagctaccgcaggatctgtgagaagaatgccaaatgatcacagttccctgtgacaagaactatacttgcaactctttttgaatccataacaggtcgtactggccaatgattacttttacttacctatctgtactaccagtagcggtccttgcccatttgggaaactgagcttctttcttctgcactgggggactggatgctagccatctccaggagacaggatcagttttacggaaacaactcagttagtatagagatgaggtccgcttctgtagtaccttccttcaaataaagaaatttggtacctgcccgg

SEQ ID NO: 2 - protein, 188 aa, Homo sapiens
  DOMAIN (26)..(188): extracellular domain
MNWHMIISGLIVVVLKVVGMTLFLLYFPQIFNKSNDGFTTTRSYGTVSQIFGSSSPSPNGFITTRSYGTVCPKDWEFYQARCFFLSTSESSWNESRDFCKGKGSTLAIVNTPEKLKFLQDITDAEKYFIGLIYHREEKRWRWINNSVFNGNVTNQNQNFNCATIGLTKTFDAASCDISYRRICEKNAK

SEQ ID NO: 3 - DNA, 896 bp, Mus musculus
aggacattaccgagcaggagcatacatttccagagcaaggagccctgctcgctgcaccgaatatcttatcaaaaagactcctatctgtatgccaacccagacttcccagaagagatcagatccctgatcccccatcatcatgaactggcacatgatcatctcggggcttatcgtagtagtgatcaaagttgttggaatgaccttttttctgctgtatttcccacaggtttttggcaaaagtaatgatggcttcgtccccacggagagctacggaaccactagtgtgcagaatgtctcacagatctttgggagaaatgacgaaagtaccatgcctacaaggagctatggaacagtctgtcccagaaactgggattttcaccaaggaaaatgctttttcttctccttctccgaatcaccttggaaagacagcatggattattgtgcaacacaaggatccacactggcaattgtcaacactccagagaaactgaagtatcttcaggacatagctggtattgagaattactttattggtttggtacgtcagcctggagagaaaaagtggcgctggatcaacaactctgtgttcaatggcaatgttaccaatcaggaccagaacttcgactgtgtcactataggtctgacgaagacatatgatgctgcatcatgtgaagtcagctatcgctggatctgcgaaatgaatgccaaatgatcatagatctctacaagagtgaatttttacagagctagcaaaggagattagttgtgactgaaaccagcccaggaaaatatagagcatcaaagactgtgcccatcttcataggtgggagttccctattgaatcctcaaagtcaattttgttactccacaaacatcttaccatagtaaaactccct

SEQ ID NO: 4 - protein, 190 aa, Mus musculus
  DOMAIN (26)..(190): extracellular domain
MNWHMIISGLIVVVIKVVGMTFFLLYFPQVFGKSNDGFVPTESYGTTSVQNVSQIFGRNDESTMPTRSYGTVCPRNWDFHQGKCFFFSFSESPWKDSMDYCATQGSTLAIVNTPEKLKYLQDIAGIENYFIGLVRQPGEKKWRWINNSVFNGNVTNQDQNFDCVTIGLTKTYDAASCEVSYRWICEMNAK

SEQ ID NO: 5 - DNA, 22 bp, Artificial
  Primer
aacggcttcattaccacaagga

SEQ ID NO: 6 - DNA, 26 bp, Artificial
  Primer
ccaagatgattcagaagtggataaga

(19) United States
(12) Patent Application Publication
(10) Pub. No.: US 2006/0099144 A1
(43) Pub. Date: May 11, 2006

(54) Tumor association of MDL-1 and methods

(75) Inventors: Terrill K. McClanahan, Sunnyvale, CA (US); Joseph H. Phillips JR., Palo Alto, CA (US)

Correspondence Address: DNAX RESEARCH, INC.;LEGAL DEPARTMENT, 901 CALIFORNIA AVENUE, PALO ALTO, CA, 94304, US

(73) Assignee: Schering Corporation, Kenilworth, NJ (US)

(21) Appl. No.: 11/268,890

(22) Filed: Nov. 7, 2005

Related U.S. Application Data
(60) Provisional application No. 60/625,829, filed on Nov. 8, 2004.

(51) Int. Cl. A61K 51/00; A61K 39/395; A61K 48/00; A61K 49/16

(52) U.S. Cl. 424/1.49; 424/155.1; 514/44; 424/9.36

(57) ABSTRACT

The invention provides methods for detecting, diagnosing, localizing and imaging tumors. The invention also relates to methods for detecting, diagnosing, localizing, imaging and treating cancer.

This filing is a U.S. Patent Application which claims benefit of U.S. Provisional Patent Application No. 60/625829, filed Nov. 8, 2004, which is incorporated herein by reference.

### FIELD OF THE INVENTION

The invention relates to new methods for detecting, diagnosing, localizing and imaging tumors, particularly solid, skin (e.g., melanoma), ovarian, breast, colorectal, renal and stomach tumors. The invention also relates to new methods for detecting, diagnosing, imaging and treating cancers, particularly skin (e.g., melanoma), ovarian, breast, colorectal, renal and stomach cancers.

### BACKGROUND OF THE INVENTION

Several receptor complexes that play a role in monocytic activation and inflammatory responses (Gingras et al. (2001) Mol. Immun.38:817-824) are formed by the non-covalent association of the transmembrane adaptor glycoprotein DAP12 with receptors of the Ig superfamily (Bouchon et al. (2000) J. Immunol. 164:4991-4995; Dietrich et al. (2000) J. Immunol. 164:9-12) or the C-type lectin superfamily (Bakker et al. (1999) PNAS U.S.A. 96:9792-9796). These associations are formed by the interaction of a negatively charged amino acid residue (aspartic acid) located in the DAP12 transmembrane domain with a positively charged amino acid residue (lysine) located in the transmembrane domain of these receptors (Gingras et al. (2001) Mol. Immun.38:817-824).

DAP12 is a disulfide-bonded, homodimeric type I transmembrane glycoprotein containing an immunoreceptor tyrosine-based activation motif (ITAM) located in it's intracellular domain (Lanier, et al. (1998) Nature 391:703-707; WO 99/06557; Campbell and Colonna (1999) Int. J. Biochem. Cell Biol. 31:631-636; Lanier and Bakker (2000) Immunol. Today 21:611-614). The importance of DAP12 relies on the ITAM domain (Gingras et al. (2001) Mol. Immun.38:817-824). Because the intracellular domain of the receptors of the Ig superfamily (Bouchon et al. (2000) J. Immunol. 164: 4991-4995; Dietrich et al. (2000) J. Immunol. 164:9-12) and the C-type lectin superfamily (Bakker et al. (1999) PNAS U.S.A. 96:9792-9796) that non-covalently associate with DAP12 are too short to allow interaction with other molecules, the DAP12 cytoplasmic domain constitutes the signaling subunit of these receptor complexes. Upon engagement of the receptor ligand-binding subunit, the DAP12 cytoplasmic ITAM is phosphorylated by Src kinases. The ITAM of DAP12 then interacts with Syk cytoplasmic tyrosine kinases, which initiates a cascade of events that leads to activation (Lanier et al. (1998) Nature 391:703-707; Campbell and Colonna (1999) Int. J. Biochem. Cell Biol. 31:631-636; Lanier and Bakker (2000) Immunol. Today 21:611-614).

DAP12 is expressed in monocytes, macrophages, natural killer (NK) cells, granulocytes, dendritic cells and mast cells, where it provides signaling function for at least eight distinct receptors (Gingras et al. (2001) Mol. Immun. 38:817-824; Lanier and Bakker, (2000) Immunol. Today 21:611-614). The monocytic receptor of the C-type lectin superfamily associated with DAP12 is myeloid DAP12-associating lectin-1 (MDL-1), a type II transmembrane protein. MDL-1 was the first DAP12 associating molecule to be identified and cloned (Bakker et al. (1999) PNAS USA 96(17):9792-9796). It is expressed exclusively in monocytes and macrophages (Bakker et al. (1999) PNAS U.S.A. 96:9792-9796). The presence of a negatively charged residue in the transmembrane domain of DAP12 precludes its cell surface expression in the absence of a partner receptor, such as MDL-1, which has a positively charged residue in its transmembrane domain. However, DAP12 alone is not sufficient for its expression and function at the cell surface. Thus, the combination of a DAP12-associating molecule, such as MDL-1, and DAP12 may account for transmitting a particular physiological signal via DAP12 (Nochi et al. (2003) Am. J. of Pathology 162:1191-1201).

Tumor-infiltrating leukocytes (e.g., myeloid lineage cells or macrophages) are white blood cells that have left the blood stream and migrated into a tumor. Macrophages are a major component of the leukocyte infiltrate of tumors. Tumor-associated macrophages (TAMs) have complex dual functions in their interaction with neoplastic cells, and evidence suggests that they are part of inflammatory circuits that promote tumor progression (Mantovani et al., (1992) Immunol. Today 13:265-270; Mantovani et al., (2002) TRENDS in Immunol. 23:549-555). TAMs are reportedly a polarized M2 macrophage population. By expressing properties of polarized M2 cells, TAMs participate in circuits that regulate tumor growth and progression, adaptive immunity, stroma formation and angiogenesis. In particular, TAMS are a component of inflammatory circuits that promote tumor progression and metastasis (Mantovani et al., supra).

Cancer is the second leading cause of death in the United States (American Cancer Society Statistics 2004). Currently, one in four deaths in the United States is due to cancer (Jemal et al., (2004) CA Cancer J. Clin 54:8-29). Cancer is more easily and successfully treated when it is diagnosed early at a localized stage, rather than when at a regional or distant stage (Jemal et al., (2004) CA Cancer J. Clin 54:8-29). Treatment options include surgery, chemotherapy, radiotherapy and immunotherapy. The major modalities of therapy are either surgery or radiotherapy for both local and local-regional cancers while chemotherapy is best for systemic sites. In many clinically diagnosed solid tumors, surgical removal is considered the primary means of treatment.

The currently available methods for cancer therapy have either been of limited success in preventing recurrence or these methods have given rise to serious and undesirable side effects. Furthermore, the development of methods that permit rapid and accurate detection of cancer continues to challenge the medical community. In light of the widespread number of cancer-related deaths, as well as the inadequacies of currently available detection and treatment methods, there is a need for more effective compounds to detect and treat cancer. Thus, a need exists for methods to detect tumors, as well as a need to treat cancers.

### SUMMARY OF THE INVENTION

The present invention addresses the foregoing needs by providing methods for detecting, diagnosing, localizing and imaging cancer by detecting increased levels of MDL-1 expression, which is associated with tumors, particularly solid tumors from patients with skin (e.g., melanoma), ovarian, breast, colorectal, renal and stomach cancers. The invention also addresses these needs by providing methods for detecting, diagnosing, localizing and imaging tumors, particularly solid, skin (e.g., melanoma), ovarian, breast, colorectal, renal and stomach tumors. Further, this invention addresses these needs by providing methods for diagnosing, localizing, imaging and treating cancer using antibodies or antigen-binding fragments thereof that bind MDL-1. In addition, this invention addresses these needs by providing methods for diagnosing, localizing, imaging and treating cancer, e.g., skin (e.g., melanoma), ovarian, breast, colorectal, renal and stomach, using soluble MDL-1 protein or fragments thereof.

The present invention provides a method for diagnosing cancer comprising: (a) measuring levels of myeloid DAP12-associating lectin-1 (MDL-1) expression in a cell or tissue; and (b) comparing measured levels of MDL-1 with expression levels of MDL-1 in a cell or tissue from a control, wherein an increase in measured levels of MDL-1 expression compared to the control is associated with cancer. Also provided is the above method wherein the MDL-1 is a polypeptide or a nucleic acid. Also provided is the above method wherein the MDL-1 has an amino acid sequence at least 90% identical to SEQ ID NO: 2; or wherein the MDL-1 comprises an amino acid sequence of SEQ ID NO: 2; as well as the above method wherein the MDL-1 is present on tumor-infiltrating leukocytes within a tumor. Also provided are the above methods wherein the cancer is selected from the group consisting of melanoma, ovarian cancer, breast cancer, colorectal cancer, renal cancer and stomach cancer.

Yet another embodiment of the present invention provides a method for detecting a tumor comprising: (a) measuring levels of MDL-1 expression in a cell or tissue; and (b) comparing measured levels of MDL-1 with expression levels of MDL-1 in a cell or tissue from a control, wherein an increase in measured levels of MDL-1 expression compared to the control is associated with the presence of a tumor. Also provided is the above method wherein the MDL-1 is a polypeptide or a nucleic acid. Also provided is the above method wherein the MDL-1 has an amino acid sequence at least 90% identical to SEQ ID NO: 2; or wherein the MDL-1 comprises an amino acid sequence of SEQ ID NO: 2; as well as the above method wherein the MDL-1 is present on tumor-infiltrating leukocytes within a tumor. Also provided are the above methods wherein the tumor is selected from the group consisting of a solid tumor, a melanoma, an ovarian tumor, a breast tumor, a colorectal tumor, a renal tumor and a stomach tumor.

In another embodiment, the invention provides a method for diagnosing a tumor in a patient comprising: (a) administering to the patient an antibody or an antigen-binding fragment thereof that binds MDL-1; (b) measuring a level of binding of the antibody or the antigen-binding fragment thereof in a cell or tissue of the patient; and (c) comparing the measured level of binding with a level of binding of an antibody or an antigen-binding fragment thereof that binds to MDL-1 in a cell or tissue of a control, wherein an increase in measured levels of binding in the patient compared to the control is associated with the presence of a tumor. Also provided is the above method wherein the MDL-1 has an amino acid sequence at least 90% identical to SEQ ID NO: 2; or wherein the MDL-1 comprises an amino acid sequence of SEQ ID NO: 2; as well as the above method, wherein the MDL-1 is present on tumor-infiltrating leukocytes within a tumor. Also provided are the above methods, wherein the tumor is selected from the group consisting of a solid tumor, a melanoma, an ovarian tumor, a breast tumor, a colorectal tumor, a renal tumor and a stomach tumor; as well as the above methods wherein the antibody or the antigen-binding fragment thereof is selected from the group consisting of a monoclonal antibody, a polyclonal antibody, an activating antibody, an inhibitory antibody, a chimeric antibody, a humanized antibody, a diabody, a single-chain antibody and a fusion protein; or the above-methods wherein the antigen-binding fragment is selected from the group consisting of a Fab fragment, a F(ab)₂ fragment, and a Fv fragment; or the above methods wherein the antibody or antigen-binding fragment thereof is bound to a label; or the above methods wherein the label is selected from the group consisting of a radiolabel, a fluorescent label, a chemiluminescent label, a paramagnetic label and an enzymatic label.

In another embodiment, the invention provides a method for detecting a tumor comprising: (a) administering to a patient an antibody or an antigen-binding fragment thereof that binds MDL-1; (b) measuring a level of binding of the antibody or antigen-binding fragment thereof in a cell or tissue of the patient; and (c) comparing the measured level of binding with a level binding of an antibody or an antigen-binding fragment thereof that binds MDL-1 in a cell or tissue of a control, wherein an increase in measured levels of binding of the antibody or antigen-binding fragment thereof in the patient compared to the control is associated with the presence of a tumor. Also provided is the above method wherein the MDL-1 has an amino acid sequence at least 90% identical to SEQ ID NO: 2; or wherein the MDL-1 comprises an amino acid sequence of SEQ ID NO: 2; as well as the above method, wherein the MDL-1 is present on tumor-infiltrating leukocytes within a tumor. Also provided are the above methods, wherein the tumor is selected from the group consisting of a solid tumor, a melanoma, an ovarian tumor, a breast tumor, a colorectal tumor, a renal tumor and a stomach tumor; or the above methods wherein the antibody or the antigen-binding fragment thereof is selected from the group consisting of a monoclonal antibody, a polyclonal antibody, an activating antibody, an inhibitory antibody, a chimeric antibody, a humanized antibody, a diabody, a single-chain antibody and a fusion protein; or the above methods wherein the antigen-binding fragment is selected from the group consisting of a Fab fragment, a F(ab)₂ fragment, and a Fv fragment; or the above methods wherein the antibody or the antigen-binding fragment thereof is bound to a label; or the above methods wherein the label is selected from the group consisting of a radiolabel, a fluorescent label, a chemiluminescent label, a paramagnetic label and an enzymatic label.

In another embodiment, the invention provides a method for treating cancer comprising administering to a patient a composition comprising an antibody or an antigen-binding fragment thereof that binds MDL-1, wherein the antibody or the antigen-binding fragment thereof is bound to a cytotoxic agent. Also provided is the above method wherein the MDL-1 has an amino acid sequence at least 90% identical to SEQ ID NO: 2; or wherein the MDL-1 comprises an amino acid sequence of SEQ ID NO: 2; as well as the above method, wherein the MDL-1 is present on tumor-infiltrating leukocytes within a tumor. Also provided are methods wherein the cancer is selected from the group consisting of melanoma, ovarian cancer, breast cancer, colorectal cancer, renal cancer and stomach cancer; or the above methods wherein the antibody or the antigen-binding fragment thereof is selected from the group consisting of a monoclonal antibody, a polyclonal antibody, an activating antibody, an inhibitory antibody, a chimeric antibody, a humanized antibody, a diabody, a single-chain antibody and a fusion protein; or the above methods wherein the antigen-binding fragment is selected from the group consisting of a Fab fragment, a F(ab)₂ fragment, and a Fv fragment. Also provided are the above methods wherein the cytotoxic agent is selected from the group consisting of a drug; a toxin; a compound that emits radiation; a molecule of plant, fungal, or bacterial origin; a biological protein; and mixtures thereof; or the above-methods wherein the compound that emits radiation is an α-emitter, a β-emitter, or a γ-emitter; or the above methods wherein the composition ablates tumor cells, kills tumor cells or reduces tumor size.

In another embodiment, the invention provides a method for treating cancer comprising administering to a patient a composition comprising a soluble MDL-1 polypeptide of a fragment thereof that binds to a ligand, wherein the polypeptide or the fragment thereof is bound to a cytotoxic agent. Also provided is the above method wherein the soluble MDL-1 polypeptide has an amino acid sequence comprising amino acid residues 26 to 188 of SEQ ID NO: 2. Also provided are methods wherein the cancer is selected from the group consisting of melanoma, ovarian cancer, breast cancer, colorectal cancer, renal cancer and stomach cancer. Also provided are the above methods wherein the cytotoxic agent is selected from the group consisting of a drug; a toxin; a compound that emits radiation; a molecule of plant, fungal, or bacterial origin; a biological protein; and mixtures thereof; or the above-methods wherein the compound that emits radiation is an α-emitter, a β-emitter, or a γ-emitter; or the above methods wherein the composition ablates tumor cells, kills tumor cells or reduces tumor size.

In another embodiment the invention provides a method for diagnosing the presence of a tumor comprising: (a) administering to a patient a soluble MDL-1 polypeptide or fragment thereof; (b) measuring a level of binding of the polypeptide or the fragment thereof to a ligand in a cell or tissue of the patient; and (c) comparing the measured level of binding with a level of binding of a soluble MDL-1 polypeptide or a fragment thereof to a ligand in a cell or tissue of a control, wherein an increase in measured levels of binding in the patient compared to the control is associated with the presence of a tumor. Also provided is the above method, wherein the soluble MDL-1 polypeptide has an amino acid sequence comprising amino acid residues 26 to 188 of SEQ ID NO: 2. Also provided are the above methods, wherein the tumor is selected from the group consisting of a solid tumor, a melanoma, an ovarian tumor, a breast tumor, a colorectal tumor, a renal tumor and a stomach tumor; or the above methods, wherein the soluble MDL-1 polypeptide or the fragment thereof is bound to a label; or the above methods wherein the label is selected from the group consisting of a radiolabel, a fluorescent label, a chemiluminescent label, a paramagnetic label and an enzymatic label.

In another embodiment, the invention provides a method for detecting a tumor in a patient comprising: (a) administering to a patient a soluble MDL-1 polypeptide or a fragment thereof; (b) measuring a level of binding of the polypeptide or the fragment thereof to a ligand in a cell or tissue of the patient; and (c) comparing the measured level of binding with a level of binding of a soluble MDL-1 polypeptide or a fragment thereof to ligand in a cell or tissue of a control, wherein an increase in measured levels of the binding in the patient compared to the control is associated with detecting the tumor. Also provided is the above method, wherein the soluble MDL-1 polypeptide has an amino acid sequence comprising amino acid residues 26 to 188 of SEQ ID NO: 2. Also provided are the above methods, wherein the tumor is selected from the group consisting of a solid tumor, a melanoma, an ovarian tumor, a breast tumor, a colorectal tumor, a renal tumor and a stomach tumor; or the above methods, wherein the soluble MDL-1 polypeptide or the fragment thereof is bound to a label; or the above methods wherein the label is selected from the group consisting of a radiolabel, a fluorescent label, a chemiluminescent label, a paramagnetic label and an enzymatic label.

### DEFINITIONS

As used herein, the term “cancer” refers to a group of cells (usually derived from a single cell) that has lost its normal control mechanisms and thus has unregulated growth. Cancerous tissues or malignancies include those of the blood and blood-forming tissues, such as leukemias and lymphomas, and solid tumors, often termed cancer. Such cancers may be carcinomas or sarcomas.

As used herein, the term “tumor” refers to an abnormal growth or mass. Tumors may be benign or cancerous (malignant). Benign tumors are not cancer. Benign tumors may be removed from the body, and then seldom grow back. Cells from a benign tumor do not spread to surrounding tissues or to other parts of the body.

As cancerous cells grow and multiply, they form a mass of cancerous tissue, that is a tumor, which invades and destroys normal adjacent tissues. Malignant tumors are cancer. Malignant tumors usually can be removed, but they may grow back. Cells from malignant tumors can invade and damage nearby tissues and organs. Also, cancer cells can break away from a malignant tumor and enter the bloodstream or lymphatic system, which is the way cancer cells spread from the primary tumor (i.e., the original cancer) to form new tumors in other organs. The spread of cancer in the body is called metastasis (What You Need to Know About Cancer—an Overview, NIH Publication No. 00-1566; posted Sep. 26, 2000, updated Sep. 16, 2002 (2002)).

As used herein, the term “solid tumor” refers to an abnormal growth or mass of tissue that usually does not contain cysts or liquid areas. Solid tumors may be benign (not cancerous) or malignant (cancerous). Different types of solid tumors are named for the type of cells that form them. Examples of solid tumors are sarcomas, carcinomas, and lymphomas. Leukemias (cancers of the blood) generally do not form solid tumors (National Cancer Institute, Dictionary of Cancer Terms).

As used herein, the term “primary cancer” refers to the original tumor or the first tumor. Cancer may begin in any organ or tissue of the body. It is usually named for the part of the body or the type of cell in which it originates (Metastatic Cancer: Questions and Answers, Cancer Facts 6.20, National Cancer Institute, reviewed Sept. 1, 2004 (2004)).

As used herein, the term “carcinoma in situ” refers to cancerous cells that are still contained within the tissue where they started to grow, and have not yet become invasive or spread to other parts of the body.

As used herein, the term “carcinomas” refers to cancers of epithelial cells, which are cells that cover the surface of the body, produce hormones, and make up glands. Examples of carcinomas are cancers of the skin, lung, colon, stomach, breast, prostate and thyroid gland.

As used herein, the term “white blood cell” refers to a blood cell that does not contain hemoglobin. A white blood cell is also called a leukocyte. White blood cells include lymphocytes, neutrophils, eosinophils, macrophages and mast cells.

As used herein, the term “tumor-infiltrating leukocytes” (e.g., myeloid lineage cells or macrophages) refers to white blood cells that have left the blood stream and have migrated into a tumor. Thus, tumor-infiltrating leukocytes may have tumor specificity.

As used herein, the term “expression status” is used to broadly refer to the variety of factors involved in the expression, function and regulation of a gene and its products, such as the level of mRNA expression, the integrity of the expressed gene products (such as the nucleic and amino acid sequences), and transcriptional and translational modifications to these molecules.

As used herein, the term “antibody molecule” refers to whole antibodies (e.g., IgG, preferably, IgG1 or IgG4) and fragments, preferably antigen-binding fragments, thereof. Antibody fragments include Fab antibody fragments, F(ab)2 antibody fragments, Fv antibody fragments, single chain Fv antibody fragments and dsFv antibody fragments.

As used herein, the term “subject” or “patient” or “host” refers to any organism, preferably an animal, more preferably a mammal (e.g., mouse, rat, rabbit, cow, dog, cat, cow, chimpanzee, gorilla) and most preferably a human.

As used herein, the term “control” includes; a patient without cancer; a patient without a tumor; a sample from a patient without cancer; a sample from a patient without a tumor; a non cancerous sample from a patient with cancer; a non tumor sample from a patient with a tumor.

As used herein, the terms “administration” and “treatment” as it applies to an animal, human, experimental subject, cell, tissue, organ, or biological fluid, refers to contact of an exogenous pharmaceutical, therapeutic, diagnostic agent, compound, or composition to the animal, human, subject, cell, tissue, organ, or biological fluid. “Administration” and “treatment” also means in vitro, in vivo and ex vivo treatments.

As used herein, the term “therapeutically effective amount” of a therapeutic agent is defined as an amount of each active component of the pharmaceutical formulation that is sufficient to show a meaningful patient benefit, i.e., to cause a decrease in, prevention, or amelioration of the symptoms of the condition being treated. When the pharmaceutical formulation comprises a diagnostic agent, “a therapeutically effective amount” is defined as an amount that is sufficient to produce a signal, image, or other diagnostic parameter. Effective amounts of the pharmaceutical formulation will vary according to factors such as the degree of susceptibility of the individual, the age, gender, and weight of the individual, and idiosyncratic responses of the individual, see, e.g., U.S. Pat. No. 5,888,530.

As used herein, the term “exogenous” refers to substances that are produced outside an organism, cell, or human body, depending on the context. As used herein, the term “endogenous” refers to substances that are produced within a cell, organism, or human body, depending on the context.

As used herein, the term “recombinant” refers to two or more nucleic acids or proteins which are not naturally contiguous and which are fused to each other. The term may also refer to a nucleic acid or protein which has been altered (e.g., post-translationally modified or mutated) by human intervention. For example, a wild-type codon may be replaced with a redundant codon encoding the same amino acid residue or a conservative substitution, while at the same time introducing or removing a nucleic acid sequence recognition site. Similarly, nucleic acid segments encoding desired functions may be fused to generate a single genetic entity encoding a desired combination of functions not found together in nature. Although restriction enzyme recognition sites are often the targets of such artificial manipulations, other site-specific targets, e.g., promoters, DNA replication sites, regulation sequences, control sequences, or other useful features may be incorporated by design. Sequences encoding epitope tags for detection or purification, as described below, may also be incorporated.

As used herein, the term “polynucleotide”, “nucleic acid ” or “nucleic acid molecule” refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in single stranded form, double-stranded form or otherwise.

As used herein, the term “polynucleotide sequence”, “nucleic acid sequence” or “nucleotide sequence” refers to a series of nucleotide bases (also called “nucleotides”) in a nucleic acid, such as DNA or RNA, and means any chain of two or more nucleotides.

As used herein, the term “coding sequence” or a sequence “encoding” refers to an expression product, such as a RNA, polypeptide, protein, or enzyme, is a nucleotide sequence that, when expressed, results in production of the product.

As used herein, the term “gene” means a DNA sequence that codes for or corresponds to a particular sequence of ribonucleotides or amino acids which comprise all or part of one or more RNA molecules, proteins or enzymes, and may or may not include regulatory DNA sequences, such as promoter sequences, which determine, for example, the conditions under which the gene is expressed. Genes may be transcribed from DNA to RNA which may or may not be translated into an amino acid sequence.

As used herein, the term “amplification” of DNA refers to the use of polymerase chain reaction (PCR) to increase the concentration of a particular DNA sequence within a mixture of DNA sequences. For a description of PCR see Saiki, et al., Science (1988) 239: 487.

As used herein, the term “oligonucleotide” refers to a nucleic acid, generally of at least 10 e.g., 10, 11, 12, 13 or 14, preferably at least 15 e.g., 15, 16, 17, 18 or 19, and more preferably at least 20 nucleotides e.g., 20, 21, 22, 23, 24, 25, 26, 27, 28, 29 or 30, preferably no more than 100 nucleotides e.g., 40, 50, 60, 70, 80 or 90, that may be hybridizable to a genomic DNA molecule, a cDNA molecule, or an mRNA molecule encoding a gene, mRNA, cDNA, or other nucleic acid of interest. Oligonucleotides may be labeled, e.g., by incorporation of ³²P-nucleotides, 3H-nucleotides, ¹⁴C-nucleotides, ³⁵S-nucleotides or nucleotides to which a label, such as biotin, has been covalently conjugated. In one embodiment, a labeled oligonucleotide may be used as a probe to detect the presence of a nucleic acid. In another embodiment, oligonucleotides (one or both of which may be labeled) may be used as PCR primers, either for cloning full length or a fragment of the gene, or to detect the presence of nucleic acids. Generally, oligonucleotides are prepared synthetically, preferably on a nucleic acid synthesizer.

As used herein, the term “promoter” or “promoter sequence” refers to a DNA regulatory region capable of binding an RNA polymerase in a cell (e.g., directly or through other promoter-bound proteins or substances) and initiating transcription of a coding sequence. A promoter sequence is, in general, bounded at its 3′ terminus by the transcription initiation site and extends upstream (5′ direction) to include the minimum number of bases or elements necessary to initiate transcription at any level. Within the promoter sequence may be found a transcription initiation site (conveniently defined, for example, by mapping with nuclease S1), as well as protein binding domains (consensus sequences) responsible for the binding of RNA polymerase. The promoter may be operably associated with other expression control sequences, including enhancer and repressor sequences or with a nucleic acid of the invention.

As used herein, the terms “express” and “expression” mean allowing or causing the information in a gene, RNA or DNA sequence to become manifest; for example, producing a protein by activating the cellular functions involved in transcription and translation of a corresponding gene. A DNA sequence is expressed in or by a cell to form an “expression product” such as an RNA (e.g., mRNA) or a protein (e.g., antibody or a fragment thereof). The expression product itself may also be said to be “expressed” by the cell.

As used herein, the terms “vector”, “cloning vector” and “expression vector” mean the vehicle (e.g., a plasmid) by which a DNA or RNA sequence may be introduced into a host cell, so as to transform the host and, optionally, promote expression and/or replication of the introduced sequence.

As used herein, the term “transfection” or “transformation” means the introduction of a nucleic acid into a cell. The introduced gene or sequence may be called a “clone”. A host cell that receives the introduced DNA or RNA has been “transformed” and is a “transformant” or a “clone”. The DNA or RNA introduced to a host cell may come from any source, including cells of the same genus or species as the host cell, or cells of a different genus or species.

As used herein, the term “host cell” means any cell of any organism that is selected, modified, transfected, transformed, grown, or used or manipulated in any way, for the production of a substance by the cell, for example the expression or replication, by the cell, of a gene, a DNA or RNA sequence, a protein or an enzyme.

As used herein, the term “expression system” means a host cell and compatible vector which, under suitable conditions, may express a protein or nucleic acid which is carried by the vector and introduced to the host cell. Common expression systems include E. coli host cells and plasmid vectors, insect host cells and Baculovirus vectors, and mammalian host cells and vectors. Suitable cells include CHO (chinese hamster ovary) cells, HeLa cells and NIH 3T3 cells and NSO cells (non-Ig-producing murine myeloma cell line). Nucleic acids encoding an antibody or antigen-binding fragment of the invention may be expressed at high levels in an E.coli/T7 expression system as disclosed in U.S. Pat. Nos. 4,952,496; 5,693,489 and 5,869,320 and in Davanloo et al., (1984) Proc. Natl. Acad. Sci. USA 81, 2035-2039; Studier et al., (1986) J. Mol. Biol. 189: 113-130; Rosenberg et al., (1987) Gene 56: 125-135; and Dunn et al., (1988) Gene 68: 259 which are herein incorporated by reference.

As used herein, the term “sequence-conservative variants” of a polynucleotide sequence refers to those in which a change of one or more nucleotides in a given codon results in no alteration in the amino acid encoded at that position. Function-conservative variants of the antibodies of the invention are also contemplated by the present invention. As used herein, the term “function-conservative variants” refers to those in which one or more amino acid residues in a protein or enzyme have been changed without altering the overall conformation and function of the polypeptide, including, but, by no means, limited to, replacement of an amino acid with one having similar properties. Amino acids with similar properties are well known in the art. For example, polar/hydrophilic amino acids which may be interchangeable include asparagine, glutamine, serine, cysteine, threonine, lysine, arginine, histidine, aspartic acid and glutamic acid; nonpolar/hydrophobic amino acids which may be interchangeable include glycine, alanine, valine, leucine, isoleucine, proline, tyrosine, phenylalanine, tryptophan and methionine; acidic amino acids which may be interchangeable include aspartic acid and glutamic acid and basic amino acids which may be interchangeable include histidine, lysine and arginine.

As used herein, the term “isolated nucleic acid” or “isolated polypeptide” may refer to a nucleic acid, such as an RNA or DNA molecule or a mixed polymer, or to a polypeptide, respectively, which is partially or fully separated from other components that are normally found in cells or in recombinant DNA expression systems. These components include, but are not limited to, cell membranes, cell walls, ribosomes, polymerases, serum components, and flanking genomic sequences. The term thus includes a nucleic acid that has been removed from its naturally occurring environment, and may include recombinant or cloned DNA isolates and chemically synthesized analogs or analogs biologically synthesized by heterologous systems. An isolated nucleic acid or polypeptide will, preferably, be an essentially homogeneous composition of molecules but may contain some heterogeneity.

As used herein, the terms “polypeptide”, “peptide” and “protein” encompass all such modifications, particularly those that are present in polypeptides synthesized by expressing a polynucleotide in a host cell.

As used herein, the term “antisense” refers to any composition containing nucleotide sequences which are complementary to a specific DNA or RNA sequence. The term “antisense strand” is used in reference to a nucleic acid strand that is complementary to the “sense” strand. Antisense molecules include peptide nucleic acids and may be produced by any method including synthesis or transcription. Once introduced into a cell, the complementary nucleotides combine with natural sequences produced by the cell to form duplexes and block either transcription or translation. The designation “negative” is sometimes used in reference to the antisense strand, and “positive” is sometimes used in reference to the sense strand.

As used herein, the term “antigenic determinant” refers to that fragment of a molecule (i.e., an epitope) that makes contact with a particular antibody. When a protein or fragment of a protein is used to immunize a host animal, numerous regions of the protein may induce the production of antibodies which bind specifically to a given region or three-dimensional structure on the protein; these regions or structures are referred to as antigenic determinants. An antigenic determinant may compete with the intact antigen (i.e., the immunogen used to elicit the immune response) for binding to an antibody.

As used herein, the term “antibody molecule” includes, but is not limited to, antibodies and fragments, preferably antigen-binding fragments, thereof. The term includes monoclonal antibodies, polyclonal antibodies, bispecific antibodies, Fab antibody fragments, F(ab)₂ antibody fragments, Fv antibody fragments (e.g., V_{H} or V_{L}), single chain Fv antibody fragments (scFv) and dsFv antibody fragments. Furthermore, the antibody molecules of the invention may be fully human antibodies or chimeric antibodies.

As used herein, the term “K_{off}” refers to the off-rate constant for dissociation of the antibody from an antibody/antigen complex.

As used herein, the term “Kₒₙ” refers to the rate at which the antibody associates with the antigen.

As used herein, the term “K_{d}” refers to the dissociation constant of a particular antibody/antigen interaction. K_{d}=K_{off}/Kₒₙ.

As used herein, the term “monoclonal antibody” refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Monoclonal antibodies are advantageous in that they may be synthesized by a hybridoma culture, essentially uncontaminated by other immunoglobulins. The modifier “monoclonal” indicates the character of the antibody as being amongst a substantially homogeneous population of antibodies, and is not to be construed as requiring production of the antibody by any particular method. As mentioned above, the monoclonal antibodies to be used in accordance with the present invention may be made by the hybridoma method first described by Kohler, et al., (1975) Nature 256: 495.

As used herein, the term “polyclonal antibody” refers to an antibody which was produced among or in the presence of one or more other, non-identical antibodies. In general, polyclonal antibodies are produced from a B-lymphocyte in the presence of several other B-lymphocytes which produced non-identical antibodies. Usually, polyclonal antibodies are obtained directly from an immunized animal.

As used herein, the term, “bispecific or bifunctional antibody” refers to an artificial hybrid antibody having two different heavy/light chain pairs and two different binding sites. Bispecific antibodies may be produced by a variety of methods including fusion of hybridomas or linking of Fab′ fragments. See, e.g., Songsivilai et al., (1990) Clin. Exp. Immunol. 79:315-321, Kostelny et al., (1992) J Immunol. 148:1547-1553. In addition, bispecific antibodies may be formed as “diabodies” (Holliger et al., (1993) PNAS USA 90:6444-6448) or as “Janusins” (Traunecker et al., (1991) EMBO J. 10:3655-3659 and Traunecker et al., (1992) Int. J. Cancer Suppl. 7:51-52).

As used herein, the term “anti-idiotypic antibodies” or “anti-idiotypes” refers to antibodies directed against the antigen-combining region or variable region (called the idiotype) of another antibody molecule. As disclosed by Jerne et al. (Jerne, N. K., (1974) Ann. Immunol. (Paris) 125c:373 and Jerne, N. K., et al., (1982) EMBO 1:234), immunization with an antibody molecule expressing a paratope (antigen-combining site) for a given antigen (e.g., an MDL-1 peptide) will produce a group of anti-antibodies, some of which share, with the antigen, a complementary structure to the paratope. Immunization with a subpopulation of the anti-idiotypic antibodies will, in turn, produce a subpopulation of antibodies or immune cell subsets that are reactive to the initial antigen.

As used herein, the term “fully human antibody” refers to an antibody which comprises human immunoglobulin protein sequences only. A fully human antibody may contain murine carbohydrate chains if produced in a mouse, in a mouse cell or in a hybridoma derived from a mouse cell. Similarly, “mouse antibody” refers to an antibody which comprises mouse immunoglobulin sequences only.

“Humanized” anti-MDL-1 peptide antibodies are also within the scope of the present invention. As used herein, the term “humanized” or “fully humanized” refers to an antibody that contains the amino acid sequences from the six complementarity-determining regions (CDRs) of the parent antibody, e.g., a mouse antibody, grafted to a human antibody framework. Humanized forms of non-human (e.g., murine or chicken) antibodies are chimeric immunoglobulins, which contain minimal sequence derived from non-human immunoglobulin. For the most part, humanized antibodies are human immunoglobulins (recipient antibody) in which residues from a complementary determining region of the recipient are replaced by residues from a complementary determining region of a non-human species (donor antibody), such as mouse, chicken, rat or rabbit, having a desired specificity, affinity and capacity. In some instances, Fv framework residues of the human immunoglobulin are also replaced by corresponding non-human residues.

As used herein, the term “partially humanized” or “chimeric” antibody means an antibody that contains heavy and light chain variable regions of, e.g., murine origin, joined onto human heavy and light chain constant regions.

An alternative to humanization is to use human antibody libraries displayed on phage or human antibody libraries contained in transgenic mice, see, e.g., Vaughan et al. (1996) Nat. Biotechnol. 14:309-314; Barbas (1995) Nature Med. 1:837-839; de Haard et al. (1999) J. Biol. Chem. 274:18218-18230; McCafferty et al. (1990) Nature 348:552-554; Clackson et al. (1991) Nature 352:624-628; Marks et al. (1991) J. Mol. Biol. 222:581-597; Mendez et al. (1997) Nature Genet. 15:146-156; Hoogenboom and Chames (2000) Immunol. Today 21:371-377; Barbas et al. (2001) Phage Display: A Laboratory Manual, Cold Spring Harbor Laboratory Press, Cold Spring Harbor, New York; Kay et al. (1996) Phage Display of Peptides and Proteins:A Laboratory Manual, Academic Press, San Diego, Calif.; de Bruin et al. (1999) Nat. Biotechnol. 17:397-399.

As used herein, the term “human” refers to antibodies containing amino acid sequences that are of 100% human origin, where the antibodies may be expressed, e.g., in a human, animal, insect, fungal, plant, bacterial, or viral host (Baca et al. (1997) J. Biol. Chem. 272:10678-10684; Clark (2000) Immunol. Today 21:397-402).

The present invention includes “chimeric antibody” which means an antibody that comprises a variable region of the present invention fused or chimerized with an antibody region (e.g., constant region) from another, non-human species (e.g., mouse, horse, rabbit, dog, cow, chicken). These antibodies may be used to modulate the expression or activity of MDL-1 in the non-human species.

As used herein, the term “human/mouse chimeric antibody” refers to an antibody which comprises a mouse variable region (V_{H} and V_{L}) fused to a human constant region.

As used herein, the term “single-chain Fv” or “sFv” antibody fragments means antibody fragment that have the V_{H} and V_{L} domains of an antibody, wherein these domains are present in a single polypeptide chain. Generally, the sFv polypeptide further comprises a polypeptide linker between the V_{H} and V_{L} domains which enables the sFv to form the desired structure for antigen binding. Techniques described for the production of single chain antibodies (U.S. Pat. Nos. 5,476,786, 5,132,405 and 4,946,778) may be adapted to produce anti-MDL-1-specific single chain antibodies. For a review of sFv see Pluckthun in The Pharmacology of Monoclonal Antibodies, vol. 13, Rosenburg and Moore eds. Springer-Verlag, N.Y., pp. 269-315 (1994).

Single chain antibodies, single domain antibodies, and bispecific antibodies are described, see, e.g., Malecki et al. (2002) Proc. Natl. Acad. Sci. USA 99:213-218; Conrath et al. (2001) J. Biol. Chem. 276:7346-7350; Desmyter et al. (2001) J. Biol. Chem. 276:26285-26290, Kostelney et al. (1992) J. Immunol. 148:1547-1553; U.S. Pat. Nos. 5,932,448; 5,532,210; 6,129,914; 6,133,426; 4,946,778.

As used herein, the terms “disulfide stabilized Fv fragments” and “dsFv” refer to antibody molecules comprising a variable heavy chain (V_{H}) and a variable light chain (V_{L}) which are linked by a disulfide bridge.

An “effective amount” of a composition of the invention may be an amount that will ameliorate one or more of the well-known parameters that characterize medical conditions caused or mediated by the MDL-1 receptor or a functional fragment thereof. By “effective amount” it is also meant the amount or concentration of antibody needed to bind to the target antigens e.g., MDL-1, expressed on the infiltrating leukocytes of the tumor to cause tumor shrinkage for surgical removal, or disappearance of the tumor.

### DETAILED DESCRIPTION OF THE INVENTION

The invention relates to methods, both quantitative and qualitative, for detecting, diagnosing, localizing and imaging tumors, particularly solid, skin (e.g., melanoma), ovarian, breast, colorectal, renal and stomach tumors by comparing levels of MDL-1 expression in a cell or tissue with MDL-1 expression levels in a control.

The present invention also relates to diagnostic assays and methods, both quantitative and qualitative, for detecting, diagnosing, locating, and imaging cancers by comparing levels of MDL-1 expression in a cell or tissue with those of MDL-1 expression in a control.

The present invention relates to diagnostic assays and methods, both quantitative and qualitative, for detecting, diagnosing, locating, and imaging cancers by detecting increased levels of MDL-1 expression, which is associated with tumors, particularly solid tumors e.g., melanoma, ovarian, breast, colorectal, renal and stomach.

The present inventions relates to the detection of elevated expression of MDL-1 in skin (e.g., melanoma), ovarian, breast, colorectal, renal and stomach cancers relative to normal controls. The expression of MDL-1 is markedly elevated or increased in tumor tissues from melanoma, ovarian, breast, colorectal, renal and stomach tumors.

Expression of MDL-1 in matched normal/tumor samples from patients with stage I, stage II, or stage III/IV melanoma; stage I, stage II, or stage III/IV ovarian cancer; stage I, stage II or stage III/IV breast cancer; stage I, stage II or stage III/IV colorectal cancer; stage I/II and stage III/IV kidney cancer; and stage I, stage II, stage III or stage IV stomach cancer show a high level of RNA expression in the tumor tissues, suggesting that MDL-1 is a useful marker for detection of skin cancer (e.g., melanoma), ovarian cancer, breast cancer, colorectal cancer, kidney (e.g., renal) cancer and stomach cancer.

It is believed that the increased levels of MDL-1 expression identified in tumors or tumor tissue is due to expression of MDL-1 on tumor-infiltrating leukocytes (e.g., that are myeloid lineage cells or macrophages) present within the tumors. The majority of MDL-1 positive leukocytes may also express the macrophage/monocyte markers CD68, CD11b and CD206.

The terms “MDL-1”, “Myeloid DAP12 associating lectin-1”, “Myeloid DAP12-associated lectin-1”, DAP-12”, “DAP12”, “DNAX Activation Protein, 12 kD” are well known in the art. The human and mouse DAP12 and MDL-1 nucleotide and polypeptide sequences are disclosed in WO 99/06557. The human MDL-1 nucleotide and amino acid sequences are defined by SEQ ID NO: 11 and SEQ ID NO: 12 of WO 99/06557, respectively. GenBank® deposits of the human MDL-1 nucleic acid sequence (AR217548) and mouse MDL-1 nucleic and amino acid sequences (AR217549 and AAN21593, respectively) are also available. Table 1 below provides the appropriate sequence identifiers.

Soluble forms of MDL-1 are also within the scope of the invention. A structural feature of the MDL-1 protein is the extracellular domain, which is defined by amino acid residues 26 to 188 of SEQ ID NO: 2 of a human MDL-1 protein, and amino acid residues 26 to 190 of SEQ ID NO: 4 of a mouse MDL-1 protein. Soluble forms of MDL-1 (i.e., soluble MDL-1 polypeptide or soluble MDL-1 protein) comprise the extracellular domain or fragments thereof. Soluble MDL-1 polypeptides may be used as therapeutics or diagnostics similar to the use of MDL-1 antibodies or antigen-binding fragments thereof.

TABLE 1
Summary of amino acid and nucleotide sequences of the invention.

| | SEQUENCE |
|---|---|
| SEQUENCE | IDENTIFIER |
| Nucleotide sequence encoding human MDL-1 | SEQ ID NO: 1 |
| (Genbank Accession No. AR217548) | |
| Amino acid sequence of human MDL-1 | SEQ ID NO: 2 |
| Nucleotide sequence encoding murine MDL-1 | SEQ ID NO: 3 |
| (Genbank Accession No. AR217549) | |
| Amino acid sequence of murine MDL-1 | SEQ ID NO: 4 |
| (Genbank Accession No. AAN21593) | |
| Nucleotide sequence of PCR Primer | SEQ ID NO: 5 |
| Nucleotide sequence of PCR Primer | SEQ ID NO: 6 |

A number of approaches to the treatment of cancer, particularly skin (e.g., melanoma), ovarian, breast, colorectal, renal and stomach cancers expressing MDL-1 are described herein. These therapeutic approaches include antibody therapy with anti-MDL-1 antibodies and therapy with soluble MDL-1 polypeptides. In addition, given its increased expression in cancer, MDL-1 or a soluble form of MDL-1 polypeptide, is useful as a diagnostic for cancer, particularly skin (e.g., melanoma), ovarian, breast, colorectal, renal and stomach cancers and, similarly, may be a marker for other cancers expressing this receptor.

Screening for cancer, the detection of tumors or cancer, and the diagnosis of cancer may encompass screening, testing and a physical examination. Diagnosis of cancer and the detection of tumors may involve both clinical and surgical procedures. Screening for cancer, the detection of tumors and the diagnosis of cancer may use scans, such as bone scans, or other imaging tests, such as computed tomography (CT) or magnetic resonance imaging (MRI) to detect the cancer or the tumor. Ultrasound scanning uses sound waves to show the structure of internal organs and is useful for identifying and determining the size of certain cancers or tumors, particularly of the kidneys, liver, pelvis, and prostate. CT scanning may be used to detect cancer or tumors in many parts of the body. Such detection is useful in diagnosing and staging cancer. MRI is an alternative to CT. With this procedure, a very powerful magnetic field generates exquisitely detailed anatomic images. Positron emission tomography (PET) may also be used to help diagnose cancer or to detect a tumor. A PET scan images a cancer by measuring biochemical processes within it. Biopsies may also be performed to confirm that an abnormality discovered on an imaging test is cancer or a tumor.

Screening for cancer before there are symptoms in the patient can be important because it may help physicians find and treat cancer early. Early stage cancer usually does not cause pain in the patient. Cancer treatment is more likely to be effective when the cancer is detected early (i.e., at an early stage; a localized stage).

Staging describes the extent or severity of an individual's cancer based on the extent of the original (primary) tumor and the extent of spread in the body. Staging is based on knowledge of the way cancer develops. Cancer cells divide and grow without control or order to form a mass of tissue, which is called a growth or tumor. As the tumor grows, it may invade nearby organs and tissues. Cancer cells can also break away from the tumor and enter the bloodstream and lymphatic system allowing them to spread from the primary site to form new tumors in other organs. The spread of cancer is called metastasis (National Cancer Institute, Staging: Questions and Answers, Fact Sheet 5.32, reviewed Jan. 6, 2004 (2004)).

The common elements considered in most stages are location of the primary tumor, tumor size and number of tumors, lymph node involvement, cell type and tumor grade, and presence or absence of metastasis. The TNM system, one of the most commonly used staging systems, is based on the extent of the tumor (T), the extent of the spread to the lymph nodes (N), and the presence of metastasis (M). A number is added to indicate the size or extent or the tumor and the extent of spread (National Cancer Institute, Staging: Questions and Answers, Fact Sheet 5.32, reviewed Jan. 6, 2004 (2004)).

Many cancer registries, such as the National Cancer Institutes Surveillance, Epidemiology, and End Results Program (SEER) use summary staging. This system is used for all types of cancer and groups cancer cases into five main categories, which are: (a) in situ, which is early cancer that is present only in the layer of cells in which it began, (b) localized, which is cancer that is limited to the organ in which it began, without evidence of spread, (c) regional, which is cancer that has spread beyond the original (primary) site to nearby nodes or organs and tissues, (d) distant, which is cancer that has spread from the primary site to distant organs or distant lymph nodes, and (e) unknown, which is used to described cancer from which there is not enough information to assign a stage (National Cancer Institute, Staging: Questions and Answers, Fact Sheet 5.32; reviewed Jan. 6, 2004 (2004)).

The types of tests for staging depend of the type of cancer. Screening and detection tests may include the following: (a) physical examinations, which are used to gather information about the cancer, (b) imaging studies, which are used to produce pictures of areas inside the body and include procedures such as x-rays, computed tomography (CT) scans, magnetic resonance (MRI) scans, and positron emission tomography (PET) scans, which can show the location of the cancer, the size of the tumor and whether the cancer has spread, (c) laboratory tests of blood, urine, other fluids and tissues taken from the body, (d) pathology reports, which may include information about the size of the tumor, the growth of the tumor, the type of cancer cells, and the grade of the tumor (how closely the cancer cells resemble normal tissue), a biopsy (the removal or cells or tissues for examination under the microscope) may be performed to provide information for the pathology report, and (e) surgical reports describe what is found during surgery such as descriptions of the size and appearance of the tumor (National Cancer Institute, Staging: Questions and Answers, Fact Sheet 5.32, reviewed Jan. 6, 2004 (2004)).

Cancer tissues may be diagnosed and staged using methods well known in the art. See, e.g., Greene et al. (eds.) (2002) AJCC Cancer Staging Manual, Springer-Verlag, New York, N.Y.

Response of solid tumors to cancer treatments or therapies may be assessed by one of skill in the art according to the guidelines described by Therasee et al. (Therasse et al., (2000) J. of the National Cancer Institute 92:205-216).

Assays that evaluate the expression level of the MDL-1 gene or MDL-1 gene products in an individual provides information on the growth or oncogenic potential of a biological sample from a patient. For example, because MDL-1 mRNA is highly expressed in melanoma, breast, ovarian, colorectal, kidney and stomach cancers, and not in matched normal tissue, assays that evaluate the relative levels of MDL-1 mRNA transcripts or proteins in a biological sample can be used to diagnose a disease associated with MDL-1 increased expression, such as cancer, and may provide prognostic information useful in defining appropriate therapeutic options.

The finding that MDL-1 mRNA is highly expressed in cancers, and not in matched normal tissue, provides evidence that this gene is associated with tumors or cancerous cell growth, and therefore identifies this gene and its products as targets that the skilled artisan can use to evaluate biological samples from individuals suspected of having a disease associated with increased expression of MDL-1.

The expression level of MDL-1 may provide information useful for predicting susceptibility to particular disease states such as progression and/or tumor aggressiveness. The invention provides methods and assays for determining MDL-1 expression level and diagnosing cancers that express MDL-1, such as cancers of the skin, breast, ovary, colon, rectum, kidney, and stomach cancers. MDL-1 expression level in patient samples may be analyzed by a number of means that are well known in the art, including without limitation, immunohistochemical analysis, in situ hybridization, RT-PCR analysis on laser capture micro-dissected samples, western blot analysis of clinical samples and cell lines, and tissue array analysis.

In another embodiment, the invention provides assays useful in determining the presence of cancer in an individual, comprising detecting an increase in MDL-1 mRNA or protein expression in a test cell or tissue sample relative to expression levels in the corresponding control cell or tissue. The presence of MDL-1 mRNA may, for example, be evaluated in tissue samples including but not limited to skin, breast, colon, rectum, ovary, kidney and stomach. The presence of elevated MDL-1 expression in any of these tissues is useful to indicate the emergence, presence and localization of these cancers, since the corresponding normal tissues express MDL-1 mRNA at lower levels.

In another embodiment, MDL-1 expression level may be determined at the protein level rather than at the nucleic acid level. For example, such a method or assay comprises determining the level of MDL-1 protein expressed by cells in a test tissue sample and comparing the level so determined to the level of MDL-1 expressed in a corresponding control sample. In one embodiment, the presence of MDL-1 protein is evaluated, for example, using immunohistochemical methods. MDL-1 antibodies or binding partners capable of detecting MDL-1 protein expression may be used in a variety of assay formats well known in the art for this purpose.

The terms “levels of MDL-1 expression”, “measured levels of MDL-1 expression” “measuring levels of MDL-1 expression” as used herein, means levels of the MDL-1 nucleic acids or polypeptides expressed in a cell or tissue. For example, the polypeptide expressed by the genes comprising the polynucleotide sequence of any of SEQ ID NOs: 1 and 3. Alternatively, the terms can mean levels of the native mRNA encoded by any of the genes comprising any of the polynucleotide sequences of, for example, SEQ ID NOs: 1 and 3 or levels of the gene comprising any of the polynucleotide sequences of for example, SEQ ID NOs: 1 and 3. Such levels are preferably measured, including determination of normal and abnormal levels, in at least one of the following from a patient: cells, tissues, and/or bodily fluids. Thus, for instance, a diagnostic assay in accordance with the invention for measuring changes in levels of MDL-1 expression or MDL-1 protein compared to normal control bodily fluids, cells, or tissue samples may be used to diagnose the presence of cancers, including ovarian cancer, breast cancer, colorectal cancer, renal cancer, stomach cancer and malignant melanoma. Further, a diagnostic assay in accordance with the invention for measuring changes in levels of MDL-1 expression or MDL-1 protein compared to normal control bodily fluids, cells, or tissue samples may be used to diagnose the presence of a tumor, including a melanoma, an ovarian tumor, a breast tumor, a colorectal tumor, a renal tumor, and a stomach tumor. In a preferred embodiment of the invention, the tumor is a solid tumor.

By “change” it is meant an increase in levels of the MDL-1 expression. For example, an increase in levels as compared to controls is associated with ovarian cancer, breast cancer, colorectal cancer, renal cancer, stomach cancer and melanoma. For example, an increase in levels as compared to controls is associated with the presence of a tumor, growth of a tumor, including an ovarian tumor, a breast tumor, a colorectal tumor, a renal tumor, a stomach tumor. In a preferred embodiment, the tumor is a solid tumor.

By “fold increase” it is generally meant that the median expression value is two times higher than the control or more, including three, or four times, five times higher than the control value or more, including six, seven, eight, or nine times, more preferably ten times higher than the control or more, including eleven, twelve, thirteen or fourteen times, fifteen times higher than the control or more, including sixteen, seventeen, eighteen, or nineteen times, twenty times higher than the control or more, including twenty-one, twenty-two, twenty-three, or twenty-four times; or twenty-five times higher than the control or more.

The increase in levels of MDL-1 expression is generally in the range of two to five times the control or more; five to ten times the control or more; fifteen to twenty times the control or more; or twenty to twenty-five times the control or more.

Methods for detecting and quantifying the expression of MDL-1 mRNA or protein are described herein and use standard nucleic acid and protein detection and quantification technologies that are well known in the art. Standard methods for the detection and quantification of MDL-1 mRNA include in situ hybridization using labeled MDL-1 riboprobes, northern blot and related techniques using MDL-1 polynucleotide probes, RT-PCR analysis using primers specific for MDL-1, and other amplification type detection methods, such as, for example, branched DNA, SISBA, TMA and the like. In a specific embodiment, semi-quantitative RT-PCR may be used to detect and quantify MDL-1 mRNA expression, as described in the Examples that follow. Any number of primers capable of amplifying MDI-1 may be used for this purpose, including, but not limited to, the various primer sets specifically described herein. Standard methods for the detection and quantification of protein may be used for this purpose. In a specific embodiment, polyclonal or monoclonal antibodies specifically reactive with the wild-type MDL-1 protein may be used in an immunohistochemical assay of biopsied tissue.

The cell surface expression of MDL-1 indicates that this molecule is an attractive target for antibody-based therapeutic strategies. Antibodies specifically reactive with extracellular domains of MDL-1 may be useful to treat MDL-1-positive cells present in tumors either as conjugates with a toxin or therapeutic agent, or as naked antibodies capable of inhibiting cell proliferation or function. Soluble forms of the receptor which comprise the extracellular domain or a fragment thereof may be useful to treat MDL-1-positive cells present in tumors either as conjugates with a toxin or therapeutic agent or as naked soluble proteins capable of inhibiting intercellular signaling by competing for the binding of the ligand or for competing with the formation of the MDL-1/DAP12 receptor complex.

MDL-1 antibodies may be introduced into a patient such that the antibody binds to MDL-1 on the cancer cells or the tumor infiltrating leukocytes present in the tumor and mediates the destruction of the cells and the tumor and/or inhibits the growth of the cells or the tumor. Mechanisms by which such antibodies exert a therapeutic effect may include complement-mediated cytolysis, antibody-dependent cellular cytotoxicity, modulating the physiological function of MDL-1, inhibiting ligand binding or signal transduction pathways, modulating tumor cell differentiation, altering tumor angiogenesis factor profiles, and/or by inducing apoptosis. MDL-1 antibodies may be conjugated to toxic or therapeutic agents and used to deliver the toxic or therapeutic agent directly to MDL-1 -bearing tumor cells or cells associated with the tumor. Examples of toxic agents include, but are not limited to, calchemicin, maytansinoids, and radioisotopes.

Cancer immunotherapy using anti-MDL-1 antibodies may follow the teachings generated from various approaches that have been successfully employed in the treatment of other types of cancer, including, but not limited to, colon cancer (Arlen et al., 1998, Crit. Rev. Immunol. 18:133-138), multiple myeloma (Ozaki et al., 1997, Blood 90:3179-3186; Tsunenari et al., 1997, Blood 90:2437-2444), gastric cancer (Kasprzyk et al., 1992, Cancer Res. 52:2771-2776), colorectal cancer (Moun et al., 1994, Cancer Res. 54:6160-6166); Velders et al., 1995, Cancer Res. 55:4398-4403), and breast cancer (Shepard et al., 1991, J. Clin. Immunol. 11:117-127).

It may be desirable for some cancer patients to be evaluated for the presence and level of MDL-1 expression, preferably using immunohistochemical assessments of tumor tissue, quantitative MDL-1 imaging, or other techniques capable of reliably indicating the presence and degree of MDL-1 expression. Immunohistochemical analysis of tumor biopsies or surgical specimens may be preferred for this purpose. Methods for immunohistochemical analysis of tumor tissues are well known in the art.

The present invention also relates to the use of antibodies or antigen-binding fragments thereof that recognize MDL-1 and to the use of soluble forms of MDL-1 or soluble MDL-1 proteins which are associated with certain solid cancerous tumors e.g., melanoma, ovarian, breast, colorectal, renal and stomach. These antibodies or antigen-binding fragments thereof or soluble MDL-1 proteins may be labeled and used for detection of cancerous tissues, particularly cancerous tissues derived from solid tumors or containing infiltrating leukocytes, which express MDL-1. The labeled antibodies or labeled soluble MDL-1 proteins may be used to detect, diagnose, localize and image tumors, particularly solid tumors, melanoma, ovarian tumors, breast tumors, colorectal tumors, renal tumors and stomach tumors, or containing infiltrating leukocytes, which express MDL-1.

The labeled antibodies or labeled soluble MDL-1 proteins may be used to detect the presence of a solid cancerous tumor. They also may be used bound to a substance effective to inhibit the growth of cells or ablate or kill cells, preferably cells associated with a tumor, both in vitro and in vivo, as therapy for cancers. It is believed that the antibodies and antigen-binding fragments of the invention target MDL-1 on tumor infiltrating leukocytes that are present in tumors. It is also believed that the soluble MDL-1 proteins of the invention target the MDL-1 ligand in tumor-infiltrating leukocytes that are present in tumors.

### Diagnostic Assays

The present invention provides methods for diagnosing the presence of cancer by analyzing expression levels of MDL-1 in test cells, tissue or bodily fluids compared with MDL-1 levels in cells, tissues or bodily fluids of preferably the same type from a control. As demonstrated herein, an increase in level of MDL-1 expression, for example, SEQ ID NO: 2, in the patient versus the control is associated with the presence of cancer. In a preferred embodiment, the cancer is associated with solid tumors (in which the tumor is a localized growth) e.g., melanoma, ovarian, breast, colorectal, renal and stomach.

Typically, for a quantitative diagnostic assay, a positive result indicating the patient tested has cancer is one in which the cells, tissues, or bodily fluids has an MDL-l expression level at least two times higher, five times higher, ten times higher, fifteen times higher, twenty times higher, twenty-five times higher.

Assay techniques that may be used to determine levels of gene expression, such as MDL-1, of the present inventions, in a sample derived from a host are well known to those of skill in the art. Such assay methods include radioimmunoassays, reverse transcriptase PCR (RT-PCR) assays, quantitative real-time PCR assays, immunohistochemistry assays, in situ hybridization assays, competitive-binding assays, western blot assays, ELISA assays, and flow cytometric assays, for example, two color FACS analysis for M2 versus M1 phenotyping of tumor-associated macrophages (Mantovani et al., (2002) TRENDS in Immunology 23:549-555).

An ELISA assay initially comprises preparing an antibody, specific to MDL-1, preferably a monoclonal antibody. In addition, a reporter antibody generally is prepared that binds specifically to MDL-1. The reporter antibody is attached to a detectable reagent such as radioactive, fluoresecent or an enzymatic reagent, for example horseradish peroxidase enzyme or alkaline phosphatase.

To carry out the ELISA, antibody specific to MDL-1 is incubated on a solid support, e.g., a polystyrene dish that binds the antibody. Any free protein binding sites on the dish are then covered by incubating with a non-specific protein, such as bovine serum albumin. Next, the sample to be analyzed is incubated in the dish, during which time MDL-1 binds to the specific antibody attached to the polystyrene dish. Unbound sample is washed out with buffer. A reporter antibody specifically directed to MDL-1 and linked to horseradish peroxidase is placed in the dish resulting in binding of the reporter antibody to any monoclonal antibody bound to MDL-1. Unattached reporter antibody is then washed out. Reagents for peroxidase activity, including a calorimetric substrate are then added to the dish. Immobilized peroxidase, linked to MDL-1 antibodies, produces a colored reaction product. The amount of color developed in a given time period is proportional to the amount of MDL-1 protein present in the sample. Quantitative results typically are obtained by reference to a standard curve.

A competition assay may be employed wherein antibodies specific to MDL-1 are attached to a solid support and labeled MDL-1 and a sample derived from the host are passed over the solid support and the amount of label detected attached to the solid support can be correlated to a quantity of MDL-1 in the sample.

Using all or a portion of a nucleic acid sequence of MDL-1 of the present invention as a hybridization probe, nucleic acid methods may also be used to detect levels of MDL-1 mRNA as tumor marker including a melanoma, an ovarian tumor, a breast tumor, a colorectal tumor, a renal tumor, and a stomach tumor. Polymerase chain reaction (PCR) and other nucleic acid methods, such as ligase chain reaction (LCR) and nucleic acid sequence based amplification (NASBA), may be used to detect cells for diagnosis and monitoring of various malignancies. For example, reverse-transcriptase PCR (RT-PCR) is a powerful technique that may be used to detect the presence of a specific mRNA population in a complex mixture of thousands of other mRNA species. In RT-PCR, an mRNA species is first reverse transcribed to complementary DNA (cDNA) with use of the enzyme reverse transcriptase; the cDNA is then amplified as in a standard PCR reaction. RT-PCR may thus reveal by amplification the presence of a single species of mRNA. Accordingly, if the mRNA is highly specific for the cell that produces it, RT-PCR may be used to identify the presence and/or absence of a specific type of cell.

Hybridization to clones or oligonucleotides arrayed on a solid support (i.e. gridding) may be used to both detect the expression of and quantitate the level of expression of that gene. In this approach, all or a portion of a cDNA encoding the MDL-1 is fixed to a substrate. The substrate may be of any suitable type including, but not limited to, glass, nitrocellulose, nylon or plastic. At least a portion of the DNA encoding the MDL-1 is attached to the substrate and then incubated with the analyte, which may be RNA or a complementary DNA (cDNA) copy of the RNA, isolated from the tissue of interest. Hybridization between the substrate bound DNA and the analyte may be detected and quantitated by several means including, but not limited to, radioactive labeling or fluorescence labeling of the analyte or a secondary molecule designed to detect the hybrid. Quantitation of the level of gene expression may be done by comparison of the intensity of the signal from the analyte compared with that determined from known standards. The standards may be obtained by in vitro transcription of the target gene, quantitating the yield, and then using that material to generate a standard curve.

The above tests may be carried out on samples derived from a variety of cells, bodily fluids and/or tissue extracts such as homogenates or solubilized tissue obtained from a patient. Tissue extracts are obtained routinely from tissue biopsy and autopsy material. Bodily fluids useful in the present invention include blood, urine, saliva or any other bodily secretion or derivative thereof. The term “blood” is meant to include whole blood, plasma, serum or any derivative of blood.

### In Vivo Targeting of MDL-1/Cancer Therapy

Identification of MDL-1 association with solid tumors is also useful in the rational design of new therapeutics for imaging and treating cancers, and in particular cancer associated with solid tumors, such as ovarian cancer, breast cancer, colorectal cancer, renal cancer, stomach cancer and skin cancer (e.g., melanoma).

The present invention is also directed to the use of antibodies or antigen-binding fragments thereof that recognize MDL-1, such as human MDL-1, for example, SEQ ID NO: 2, which is associated with certain solid tumors e.g., melanoma, ovarian, breast, colorectal, renal and stomach. These antibodies or antigen-binding fragments thereof may be labeled and used for detection of cancerous tissues, particularly cancerous tissues derived from solid tumors or containing infiltrating leukocytes, which express MDL-1. The labeled antibodies may be used to detect the presence of a solid tumor. They also may be used bound to a substance effective to ablate or kill such cells as therapy for cancers.

For example, in one embodiment, antibodies which bind to MDL-1 may be raised and used in vivo in patients suspected of suffering from cancer. Antibodies which bind MDL-1 may be injected into a patient suspected of having cancer for diagnostic and/or therapeutic purposes. Thus, another aspect of the present invention provides for a method for the treatment of cancer in a human patient in need of such treatment by administering to the patient an effective amount of antibody.

The preparation and use of antibodies for in vivo diagnosis and treatment is well known in the art. For example, antibody-chelators labeled with Indium-111 have been described for use in the radioimmunoscintographic imaging of carcinoembryonic antigen expressing tumors (Sumerdon et al. Nucl. Med. Biol. 1990 17:247-254). In particular, these antibody-chelators have been used in detecting tumors in patients suspected of having recurrent colorectal cancer (Griffin et al. J. Clin. Onc. 1991 9:631-640). Antibodies with paramagnetic ions as labels for use in magnetic resonance imaging have also been described (Lauffer, R. B. Magnetic Resonance in Medicine 1991 22:339-342). Antibodies directed against MDL-1 may be used in a similar manner. Labeled antibodies which bind MDL-1 may be injected into patients suspected of having cancer for the purpose of diagnosing the disease status of the patient. The label used will be selected in accordance with the imaging modality to be used. For example, radioactive labels such as Indium-111, Technetium-99m or Iodine-131 may be used for planar scans or single photon emission computed tomography (SPECT). Positron emitting labels such as Fluorine-19 may be used in positron emission tomography. Paramagnetic ions such as Gadlinium (III) or Manganese (II) may be used in magnetic resonance imaging (MRI). Presence of the label, as compared to imaging of normal tissue, permits determination of the spread of the cancer. The amount of label within an organ or tissue also allows determination of the presence or absence of cancer in that organ or tissue.

### Gene Therapy

The invention further provides a means of producing the soluble form of the MDL-1 polypeptide or fragment thereof, using known techniques of gene therapy. Soluble MDL-1 polypeptide comprises the extracellular domain or a fragment thereof. Methods may be readily practiced by employing a MDL-1 soluble polypeptide or a MDL-1 soluble protein fragment or a soluble MDL-1-encoding nucleic acid molecule and recombinant vectors capable of expressing and appropriately presenting the soluble MDL-1 polypeptide.

### Kits

For use in the diagnostic and therapeutic applications described above, kits are also provided by the invention. Such kits may comprise a carrier means being compartmentalized to receive in close confinement one or more container means, such as vials, tubes, and the like, each of the container means comprising one of the separate elements to be used in the method. For example, one of the container means may comprise a probe that is or may be detectably labeled. Such probe may be an antibody or polynucleotide specific for a MDL-1 protein or a MDL-1 gene or message, respectively. Where the kit utilizes nucleic acid hybridization to detect the target nucleic acid, the kit may also have containers containing nucleotide(s) for amplification of the target nucleic acid sequence and/or a container comprising a reporter-means, such as a biotin-binding protein, such as avidin or streptavidin, bound to a reporter molecule, such as an enzymatic, fluorescent, or radioisotope label.

The kit of the invention will typically comprise the container described above and one or more other containers comprising materials desirable from a commercial and user standpoint, including buffers, diluents, filters, needles, syringes, and package inserts with instructions for use. A label may be present on the on the container to indicate that the composition is used for a specific therapy or non-therapeutic application, and may also indicate directions for either in vivo or in vitro use, such as those described above.

### Molecular Biology

In accordance with the present invention there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, New York (herein “Sambrook, et al., 1989”); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. (1985)); Transcription And Translation (B. D. Hames & S. J. Higgins, eds. (1984)); Animal Cell Culture (R. I. Freshney, ed. (1986)); Immobilized Cells And Enzymes (IRL Press, (1986)); B. Perbal, A Practical Guide To Molecular Cloning (1984); F. M. Ausubel et al. (eds.), Current Protocols in Molecular Biology, John Wiley & Sons, Inc. (1994).

The present invention includes recombinant versions of the MDL-1 antibody or antigen-binding fragment of the invention.

In a specific embodiment, the present invention includes a nucleic acid, which encodes MDL-1, a soluble MDL-1, an anti-MDL-1 antibody, an anti-MDL-1 antibody heavy or light chain, an anti-MDL-1 antibody heavy or light chain variable region, an anti-MDL-1 antibody heavy or light chain constant region or anti-MDL-1 antibody CDR (e.g., CDR-L1, CDR-L2, CDR-L3, CDR-H1, CDR-H2 or CDR-H3), which may be amplified by PCR.

The sequence of any nucleic acid (e.g., a nucleic acid encoding an MDL-1 gene or a nucleic acid encoding an anti-MDL-1 antibody or a fragment or portion thereof) may be sequenced by any method known in the art (e.g., chemical sequencing or enzymatic sequencing). “Chemical sequencing” of DNA may denote methods such as that of Maxam and Gilbert (1977) (Proc. Natl. Acad. Sci. USA 74:560), in which DNA is randomly cleaved using individual base-specific reactions. “Enzymatic sequencing” of DNA may denote methods such as that of Sanger (Sanger et al., (1977) Proc. Natl. Acad. Sci. USA 74:5463).

The nucleic acids herein may be flanked by natural regulatory (expression control) sequences, or may be associated with heterologous sequences, including promoters, internal ribosome entry sites (IRES) and other ribosome binding site sequences, enhancers, response elements, suppressors, signal sequences, polyadenylation sequences, introns, 5′- and 3′-non-coding regions, and the like.

Promoters, which may be used to control gene expression, include, but are not limited to, the cytomegalovirus (CMV) promoter (U.S. Pat. Nos. 5,385,839 and 5,168,062), the SV40 early promoter region (Benoist et al., (1981) Nature 290:304-310), the promoter contained in the 3′ long terminal repeat of Rous sarcoma virus (Yamamoto et al., (1980) Cell 22:787-797), the herpes thymidine kinase promoter (Wagner et al., (1981) Proc. Natl. Acad. Sci. USA 78:1441-1445), the regulatory sequences of the metallothionein gene (Brinster et al., (1982) Nature 296:3942); prokaryotic expression vectors such as the β-lactamase promoter (Villa-Komaroffet al., (1978) Proc. Natl. Acad. Sci. USA 75:3727-3731), or the tac promoter (DeBoer et al., (1983) Proc. Natl. Acad. Sci. USA 80:21-25); see also “Useful proteins from recombinant bacteria” in Scientific American (1980) 242:74-94; and promoter elements from yeast or other fungi such as the Gal 4 promoter, the ADC (alcohol dehydrogenase) promoter, PGK (phosphoglycerol kinase) promoter or the alkaline phosphatase promoter.

A coding sequence is “under the control of”, “functionally associated with” or “operably associated with” transcriptional and translational control sequences in a cell when the sequences direct RNA polymerase mediated transcription of the coding sequence into RNA, preferably mRNA, which then may be trans-RNA spliced (if it contains introns) and, optionally, translated into a protein encoded by the coding sequence.

The present invention contemplates modifications, especially any superficial or slight modification, to the amino acid or nucleotide sequences that correspond to the proteins e.g., MDL-1 of the invention. In particular, the present invention contemplates sequence conservative variants of the nucleic acids that encode the human MDL-1 and mouse MDL-1 of the invention.

The present invention includes MDL-1, which are encoded by nucleic acids as described in Table 1 as well as nucleic acids which hybridize thereto. Preferably, the nucleic acids hybridize under low stringency conditions, more preferably under moderate stringency conditions and most preferably under high stringency conditions and, preferably, exhibit MDL-1 activity.

A nucleic acid molecule is “hybridizable” to another nucleic acid molecule, such as a cDNA, genomic DNA, or RNA, when a single stranded form of the nucleic acid molecule may anneal to the other nucleic acid molecule under the appropriate conditions of temperature and solution ionic strength (see Sambrook et al., supra). The conditions of temperature and ionic strength determine the “stringency” of the hybridization. Typical low stringency hybridization conditions may be 55° C., 5×SSC, 0.1% SDS, 0.25% milk, and no formamide; or 30% formamide, 5×SSC, 0.5% SDS. Typical, moderate stringency hybridization conditions are similar to the low stringency conditions except the hybridization is carried out in 40% formamide, with 5× or 6×SSC. High stringency hybridization conditions are similar to low stringency conditions except the hybridization conditions are carried out in 50% formamide, 5× or 6×SSC and, optionally, at a higher temperature (e.g., 57° C., 59° C., 60° C., 62° C., 63° C., 65° C. or 68° C.). In general, SSC is 0.15M NaCl and 0.015M Na-citrate. Hybridization requires that the two nucleic acids contain complementary sequences, although, depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the higher the stringency under which the nucleic acids may hybridize. For hybrids of greater than 100 nucleotides in length, equations for calculating the melting temperature have been derived (see Sambrook et al., supra, 9.50-9.51). For hybridization with shorter nucleic acids, i.e., oligonucleotides, the position of mismatches becomes more important, and the length of the oligonucleotide determines its specificity (see Sambrook, et al., supra, 11.7-11.8).

Also included in the present invention are nucleic acids comprising nucleotide sequences and polypeptides comprising amino acid sequences that are at least 70% identical, at least 80% identical, at least 90% identical e.g., 91%, 92%, 93%, 94%, and at least 95% identical e.g., 95%, 96%, 97%, 98%, 99%, 100%, to the reference nucleotide and amino acid sequences of Table 1 when the comparison is performed by a BLAST algorithm wherein the parameters of the algorithm are selected to give the largest match between the respective sequences over the entire length of the respective reference sequences. Polypeptides comprising amino acid sequences which are at least 70% similar, at least 80% similar, at least 90% similar e.g., 91%, 92%, 93%, 94%, and at least 95% similar e.g., 95%, 96%, 97%, 98%, 99%, 100%, to the reference amino acid sequences of Table 1 e.g., SEQ ID NOs: 2 and 4, when the comparison is performed with a BLAST algorithm wherein the parameters of the algorithm are selected to give the largest match between the respective sequences over the entire length of the respective reference sequences, are also included in the present invention.

Sequence identity refers to exact matches between the nucleotides or amino acids of two sequences which are being compared. Sequence similarity refers to both exact matches between the amino acids of two polypeptides which are being compared in addition to matches between nonidentical, biochemically related amino acids. Biochemically related amino acids which share similar properties and may be interchangeable are discussed above.

The following references regarding the BLAST algorithm are herein incorporated by reference: BLAST ALGORITHMS: Altschul et al., (1990) J. Mol. Biol. 215:403-410; Gish et al., (1993) Nature Genet. 3:266-272; Madden et al., (1996) Meth. Enzymol. 266:131-141; Altschul et al., (1997) Nucleic Acids Res. 25:3389-3402; Zhang et al., (1997) Genome Res. 7:649-656; Wootton et al., (1993) Comput. Chem. 17:149-163; Hancock et al., (1994) Comput. Appl. Biosci. 10:67-70; ALIGNMENT SCORING SYSTEMS: Dayhoff et al., “A model of evolutionary change in proteins.” in Atlas of Protein Sequence and Structure, (1978) vol. 5, suppl. 3, M. O. Dayhoff (ed.), pp. 345-352, Natl. Biomed. Res. Found., Washington, DC; Schwartz et al., “Matrices for detecting distant relationships.” in Atlas of protein Sequence and Structure, (1978) vol. 5, suppl. 3, M. O. Dayhoff (ed.), pp. 353-358, Natl. Biomed. Res. Found., Washington, DC; Altschul (1991) J. Mol. Biol. 219:555-565; States et al., (1991) Methods 3:66-70; Henikoff et al., (1992) Proc. Natl. Acad. Sci. USA 89:10915-10919; Altschul et al., (1993) J. Mol. Evol. 36:290-300; ALIGNMENT STATISTICS: Karlin et al., (1990) Proc. Natl. Acad. Sci. USA 87:2264-2268; Karlin et al., (1993) Proc. Natl. Acad. Sci. USA 90:5873-5877; Dembo et al., (1994) Ann. Prob. 22:2022-2039; and Altschul, S. F. “Evaluating the statistical significance of multiple distinct local alignments.” in Theoretical and Computational Methods in Genome Research (S. Suhai, ed.), (1997) pp. 1-14, Plenum, N.Y.

The present invention also includes recombinant versions of the soluble form of MDL-1 or a fragment thereof. Soluble MDL-1 protein comprises the extracellular domain of MDL-1. Moreover, fragments of the extracellular domain will also provide soluble forms of the MDL-1 protein. Fragments can be prepared using known techniques to isolate a desired portion of the extracellular region,

The present invention also includes fusions which include the polypeptides and polynucleotides of the present invention and a second polypeptide or polynucleotide moiety, which may be referred to as a “tag”. The fusions of the present invention may comprise any of the polynucleotides or polypeptides set forth in Table 1 or any subsequence or fragment thereof. The fused polypeptides of the invention may be conveniently constructed, for example, by insertion of a polynucleotide of the invention or fragment thereof into an expression vector as described above. The fusions of the invention may include tags which facilitate purification or detection. Such tags include glutathione-S-transferase (GST), hexahistidine (His6) tags, maltose binding protein (MBP) tags, haemagglutinin (HA) tags, cellulose binding protein (CBP) tags and myc tags. Detectable labels or tags such as ³²P, ³⁵S, ¹⁴C, ³H, ^{99m}Tc, ¹¹¹In, ⁶⁸Ga, ¹⁸F, ¹²⁵I, ¹³¹I, ^{113m}In, ⁷⁶Br, ⁶⁷Ga, ^{99m}Tc, ¹²³I, ¹¹¹In and ⁶⁸Ga may also be used to label the polypeptides of the invention. Methods for constructing and using such fusions are very conventional and well known in the art.

Modifications (e.g., post-translational modifications) that occur in a polypeptide often will be a function of how it is made. For polypeptides made by expressing a cloned gene in a host, for instance, the nature and extent of the modifications, in large part, will be determined by the host cell's post-translational modification capacity and the modification signals present in the polypeptide amino acid sequence. For instance, as is well known, glycosylation often does not occur in bacterial hosts such as E. coli. Accordingly, when glycosylation is desired, a polypeptide may be expressed in a glycosylating host, generally a eukaryotic cell. Insect cells often carry out post-translational glycosylations which are similar to those of mammalian cells. For this reason, insect cell expression systems have been developed to express, efficiently, mammalian proteins having native patterns of glycosylation. Alternatively, deglycosylation enzymes may be used to remove carbohydrates attached during production in eukaryotic expression systems.

Analogs of the MDL-1 peptides of the invention may be prepared by chemical synthesis or by using site-directed mutagenesis, Gillman et al., (1979) Gene 8:81; Roberts et al., (1987) Nature, 328:731 or Innis (Ed.), 1990, PCR Protocols: A Guide to Methods and Applications, Academic Press, New York, N.Y. or the polymerase chain reaction method PCR; Saiki et al., (1988) Science 239:487, as exemplified by Daugherty et al., (1991) (Nucleic Acids Res. 19:2471) to modify nucleic acids encoding the peptides. Adding epitope tags for purification or detection of recombinant products is envisioned.

Still other analogs are prepared by the use of agents known in the art for their usefulness in cross-linking proteins through reactive side groups. Preferred derivatization sites with cross-linking agents are free amino or carboxy groups, carbohydrate moieties and cysteine residues.

### Protein Purification

Typically, the peptides of the invention may be produced by expressing a nucleic acid which encodes the polypeptide in a host cell which is grown in a culture (e.g., liquid culture such as Luria broth). For example, the nucleic acid may be part of a vector (e.g., a plasmid) which is present in the host cell. Following expression, the peptides of the invention may be isolated from the cultured cells. The peptides of this invention may be purified by standard methods, including, but not limited to, salt or alcohol precipitation, affinity chromatography (e.g., used in conjunction with a purification tagged peptide as discussed above), preparative disc-gel electrophoresis, isoelectric focusing, high pressure liquid chromatography (HPLC), reversed-phase HPLC, gel filtration, cation and anion exchange and partition chromatography, and countercurrent distribution. Such purification methods are very well known in the art and are disclosed, e.g., in “Guide to Protein Purification”, Methods in Enzymology, Vol. 182, M. Deutscher, Ed., 1990, Academic Press, New York, N.Y.

### Antibody Structure

In general, the basic antibody structural unit is known to comprise a tetramer. Each tetramer includes two identical pairs of polypeptide chains, each pair having one “light” (about 25 kDa) and one “heavy” chain (about 50-70 kDa). The amino-terminal portion of each chain may include a variable region of about 100 to 110 or more amino acids primarily responsible for antigen recognition. The carboxy-terminal portion of each chain may define a constant region primarily responsible for effector function. Typically, human light chains are classified as kappa and lambda light chains. Furthermore, human heavy chains are typically classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989)) (incorporated by reference in its entirety for all purposes).

The variable regions of each light/heavy chain pair may form the antibody binding site. Thus, in general, an intact IgG antibody has two binding sites. Except in bifunctional or bispecific antibodies, the two binding sites are, in general, the same.

Normally, the chains all exhibit the same general structure of relatively conserved framework regions (FR) joined by three hypervariable regions, also called complementarity determining regions or CDRs. The CDRs from the two chains of each pair are usually aligned by the framework regions, enabling binding to a specific epitope. In general, from N-terminal to C-terminal, both light and heavy chains comprise the domains FR1, CDR1, FR2, CDR2, FR3, CDR3 and FR4. The assignment of amino acids to each domain is, generally, in accordance with the definitions of Sequences of Proteins of Immunological Interest, Kabat et al.; National Institutes of Health, Bethesda, Md.; 5^{th} ed.; NIH Publ. No. 91-3242 (1991); Kabat (1978) Adv. Prot. Chem. 32:1-75; Kabat et al., (1977) J. Biol. Chem. 252:6609-6616; Chothia et al., (1987) J Mol. Biol. 196:901-917 or Chothia et al., (1989) Nature 342:878-883.

### Antibody Molecules

The anti-MDL-1 antibody molecules of the invention preferably recognize human MDL-1. For example, the polypeptide expressed by the genes comprising the polynucleotide sequence of SEQ ID NO: 1. For example, the soluble MDL-1 polypeptide which is defined by amino acid residues 26 to 188 of SEQ ID NO: 2 of a human MDL-1 protein. However, the present invention includes antibody molecules which recognize mouse MDL-1, and MDL-1 from other species, preferably mammals (e.g., rat, rabbit, sheep or dog). For example, the polypeptide expressed by the genes comprising the polynucleotide sequence of SEQ ID NO: 3. For example, the soluble MDL-1 polypeptide which is defined by amino acid residues 26 to 190 of SEQ ID NO: 4 of a murine MDL-1 protein. The present invention also includes anti-MDL-1 antibodies or fragments thereof which are complexed with MDL-1 or any fragment thereof or with any cell which is expressing MDL-1 or any portion or fragment thereof on the cell surface. Such complexes may be made by contacting the antibody or antibody fragment with MDL-1 or the MDL-1 fragment.

In an embodiment, fully-human monoclonal antibodies directed against MDL-1 are generated using transgenic mice carrying parts of the human immune system rather than the mouse system. These transgenic mice, which may be referred to, herein, as “HuMAb” mice, contain a human immunoglobulin gene miniloci that encodes unrearranged human heavy (μ and γ) and κ light chain immunoglobulin sequences, together with targeted mutations that inactivate the endogenous μ and κ chain loci (Lonberg, N., et al., (1994) Nature 368(6474):856-859). Accordingly, the mice exhibit reduced expression of mouse IgM or κ, and in response to immunization, the introduced human heavy and light chain transgenes undergo class switching and somatic mutation to generate high affinity human IgGκ monoclonal antibodies (Lonberg, N., et al., (1994), supra; reviewed in Lonberg, N. (1994) Handbook of Experimental Pharmacology 113:49-101; Lonberg et al., (1995) Intern. Rev. Immunol. 13:65-93, and Harding et al., (1995) Ann. N.Y Acad. Sci 764:536-546). The preparation of HuMab mice is commonly known in the art and is described, for example, in Taylor et al., (1992) Nucleic Acids Research 20:6287-6295; Chen et al., (1993) International Immunology 5:647-656; Tuaillon et al., (1993) Proc. Natl. Acad. Sci USA 90:3720-3724; Choi et al., (1993) Nature Genetics 4:117-123; Chen et al., (1993)EMBO J. 12:821-830; Tuaillon et al., (1994) J Immunol. 152:2912-2920; Lonberg et al., (1994) Nature 368(6474):856-859; Lonberg, N. (1994) Handbook of Experimental Pharmacology 113:49-101; Taylor et al., (1994) International Immunology 6:579-591; Lonberg et al., (1995) Intern. Rev. Immunol. Vol. 13:65-93; Harding et aL, (1995) Ann. N.Y Acad. Sci 764:536-546; Fishwild et al., (1996) Nature Biotechnology 14:845-851 and Harding et al., (1995) Annals NY Acad. Sci. 764:536-546; the contents of all of which are hereby incorporated by reference in their entirety. See further, U.S. Pat. Nos. 5,545,806; 5,569,825; 5,625,126; 5,633,425; 5,789,650; 5,877,397; 5,661,016; 5,814,318; 5,874,299; 5,770,429 and 5,545,807; and International Patent Application Publication Nos. WO 98/24884; WO 94/25585; WO 93/12227; WO 92/22645 and WO 92/03918 the disclosures of all of which are hereby incorporated by reference in their entity.

To generate fully human, monoclonal antibodies to MDL-1, HuMab mice may be immunized with an antigenic MDL-1 polypeptide as described by Lonberg et al., (1994) Nature 368(6474):856-859; Fishwild et al., (1996) Nature Biotechnology 14:845-851 and WO 98/24884. Preferably, the mice will be 6-16 weeks of age upon the first immunization. For example, a purified preparation of MDL-1 may be used to immunize the HuMab mice intraperitoneally. The mice may also be immunized with whole cells which are stably transformed or transfected with an MDL-1 gene.

In general, HuMAb transgenic mice respond well when initially immunized intraperitoneally (IP) with antigen in complete Freund's adjuvant, followed by every other week IP immunizations (usually, up to a total of 6) with antigen in incomplete Freund's adjuvant. Mice may be immunized, first, with cells expressing MDL-1, then with a soluble fragment of MDL-1 and continually receive alternating immunizations with the two antigens. The immune response may be monitored over the course of the immunization protocol with plasma samples being obtained by retroorbital bleeds. The plasma may be screened for the presence of anti-MDL-1 antibodies, for example by ELISA, and mice with sufficient titers of immunoglobulin may be used for fusions. Mice may be boosted intravenously with antigen 3 days before sacrifice and removal of the spleen. It is expected that 2-3 fusions for each antigen may need to be performed. Several mice may be immunized for each antigen. For example, a total of twelve HuMAb mice of the HCO7 and HCO12 strains may be immunized.

Hybridoma cells which produce the monoclonal anti-MDL-1 antibodies may be produced by methods which are commonly known in the art. These methods include, but are not limited to, the hybridoma technique originally developed by Kohler, et al., (1975) (Nature 256:495-497), as well as the trioma technique (Hering et al., (1988) Biomed. Biochim. Acta. 47:211-216 and Hagiwara et al., (1993) Hum. Antibod. Hybridomas 4:15), the human B-cell hybridoma technique (Kozbor et al., (1983) Immunology Today 4:72 and Cote et al., (1983) Proc. Natl. Acad. Sci. U.S.A 80:2026-2030), and the EBV-hybridoma technique (Cole et al., in Monoclonal Antibodies and Cancer Therapy, Alan R. Liss, Inc., pp. 77-96, 1985). Preferably, mouse splenocytes are isolated and fused with PEG to a mouse myeloma cell line based upon standard protocols. The resulting hybridomas may then be screened for the production of antigen-specific antibodies. For example, single cell suspensions of splenic lymphocytes from immunized mice may by fused to one-sixth the number of P3X63-Ag8.653 nonsecreting mouse myeloma cells (ATCC, CRL 1580) with 50% PEG. Cells may be plated at approximately 2×10⁵ cells/mL in a flat bottom microtiter plate, followed by a two week incubation in selective medium containing 20% fetal Clone Serum, 18% “653” conditioned media, 5% origen (IGEN), 4 mM L-glutamine, 1 mM L-glutamine, 1 mM sodium pyruvate, 5 mM HEPES, 0.055 mM 2-mercaptoethanol, 50 units/ml penicillin, 50 mg/ml streptomycin, 50 mg/ml gentamycin and 1×HAT (Sigma; the HAT is added 24 hours after the fusion). After two weeks, cells may be cultured in medium in which the HAT is replaced with HT. Individual wells may then be screened by ELISA for human anti-MDL-1 monoclonal IgG antibodies. Once extensive hybridoma growth occurs, medium may be observed usually after 10-14 days. The antibody secreting hybridomas may be replated, screened again, and if still positive for human IgG, anti-MDL-1 monoclonal antibodies, may be subcloned at least twice by limiting dilution. The stable subclones may then be cultured in vitro to generate small amounts of antibody in tissue culture medium for characterization.

The anti-MDL-1 antibody molecules of the present invention may also be produced recombinantly (e.g., in an E.coli/T7 expression system as discussed above). In this embodiment, nucleic acids encoding the antibody molecules of the invention (e.g., V_{H} or V_{L}) may be inserted into a pET-based plasmid and expressed in the E.coli/T7 system. There are several methods by which to produce recombinant antibodies which are known in the art. One example of a method for recombinant production of antibodies is disclosed in U.S. Pat. No. 4,816,567 which is herein incorporated by reference. Transformation may be by any known method for introducing polynucleotides into a host cell. Methods for introduction of heterologous polynucleotides into mammalian cells are well known in the art and include dextran-mediated transfection, calcium phosphate precipitation, polybrene-mediated transfection, protoplast fusion, electroporation, encapsulation of the polynucleotide(s) in liposomes, biolistic injection and direct microinjection of the DNA into nuclei. In addition, nucleic acid molecules may be introduced into mammalian cells by viral vectors. Methods of transforming cells are well known in the art. See, for example, U.S. Pat. Nos. 4,399,216; 4,912,040; 4,740,461 and 4,959,455.

Mammalian cell lines available as hosts for expression are well known in the art and include many immortalized cell lines available from the American Type Culture Collection (ATCC). These include, inter alia, Chinese hamster ovary (CHO) cells, NSO, SP2 cells, HeLa cells, baby hamster kidney (BHK) cells, monkey kidney cells (COS), human hepatocellular carcinoma cells (e.g., Hep G2), A549 cells, 3T3 cells, and a number of other cell lines. Mammalian host cells include human, mouse, rat, dog, monkey, pig, goat, bovine, horse and hamster cells. Cell lines of particular preference are selected through determining which cell lines have high expression levels. Other cell lines that may be used are insect cell lines, such as Sf9 cells, amphibian cells, bacterial cells, plant cells and fungal cells. When recombinant expression vectors encoding the heavy chain or antigen-binding fragment thereof, the light chain and/or antigen-binding fragment thereof are introduced into mammalian host cells, the antibodies are produced by culturing the host cells for a period of time sufficient to allow for expression of the antibody in the host cells or, more preferably, 5 secretion of the antibody into the culture medium in which the host cells are grown.

Antibodies may be recovered from the culture medium using standard protein purification methods. Further, expression of antibodies of the invention (or other moieties therefrom) from production cell lines may be enhanced using a number of known techniques. For example, the glutamine synthetase gene expression system (the GS system) is a common approach for enhancing expression under certain conditions. The GS system is discussed in whole or part in connection with European Patent Nos. 0 216 846, 0 256 055, and 0 323 997 and European Patent Application No. 89303964.4.

It is likely that antibodies expressed by different cell lines or in transgenic animals will have different glycosylation from each other. However, all antibodies encoded by the nucleic acid molecules provided herein, or comprising the amino acid sequences provided herein are part of the instant invention, regardless of the glycosylation of the antibodies.

Antibody fragments, preferably antigen-binding antibody fragments, fall within the scope of the present invention also include F(ab)₂ fragments which may be produced by enzymatic cleavage of an IgG by, for example, pepsin. Fab fragments may be produced by, for example, reduction of F(ab)₂ with dithiothreitol or mercaptoethylamine. A Fab fragment is a V_{L}-C_{L} chain appended to a V_{H}-C_{H1} chain by a disulfide bridge. A F(ab)₂ fragment is two Fab fragments which, in turn, are appended by two disulfide bridges. The Fab portion of an F(ab)₂ molecule includes a portion of the F_{c} region between which disulfide bridges are located.

As is well known, Fv, the minimum antibody fragment which contains a complete antigen recognition and binding site, consists of a dimer of one heavy and one light chain variable domain (V_{H}-V_{L}) in non-covalent association. In this configuration that corresponds to the one found in native antibodies the three complementarity determining regions (CDRs) of each variable domain interact to define an antigen binding site on the surface of the V_{H}-V_{L} dimer. Collectively, the six CDRs confer antigen binding specificity to the antibody. Frameworks (FRs) flanking the CDRs have a tertiary structure that is essentially conserved in native immunoglobulins of species as diverse as human and mouse. These FRs serve to hold the CDRs in their appropriate orientation. The constant domains are not required for binding function, but may aid in stabilizing V_{H}-V_{L} interaction. Even a single variable domain (or half of an Fv comprising only three CDRs specific for an antigen) has the ability to recognize and bind antigen, although usually at a lower affinity than an entire binding site (Painter, Biochem. 11 (1972), 1327-1337). Hence, said domain of the binding site of the antibody construct as defined and described in the present invention may be a pair of V_{H}-V_{L}, V_{H}-V_{H} or V_{L}-V_{L} domains of different immunoglobulins. The order of V_{H} and V_{L} domains within the polypeptide chain is not decisive for the present invention, the order of domains given hereinabove may be reversed usually without any loss of function. It is important, however, that the V_{H} and V_{L} domains are arranged so that the antigen binding site may properly fold. An Fᵥ fragment is a V_{L} or V_{H} region.

Depending on the amino acid sequences of the constant domain of their heavy chains, immunoglobulins may be assigned to different classes. There are at least five major classes of immunoglobulins: IgA, IgD, IgE, IgG and IgM, and several of these may be further divided into subclasses (isotypes), e.g. IgG-1, IgG-2, IgG-3 and IgG-4; IgA-1 and IgA-2.

The anti-MDL-1 antibody molecules or the MDL-1 soluble proteins of the invention may also be conjugated to a chemical moiety. The chemical moiety may be, inter alia, a polymer, a radionuclide or a cytotoxic factor. Preferably the chemical moiety is a polymer which increases the half-life of the antibody molecule in the body of a subject. Suitable polymers include, but are not limited to, polyethylene glycol (PEG) (e.g., PEG with a molecular weight of 2 kDa, 5 kDa, 10 kDa, 12 kDa, 20 kDa, 30 kDa or 40 kDa), dextran and monomethoxypolyethylene glycol (mPEG). Lee et al., (1999) (Bioconj. Chem. 10:973-981) discloses PEG conjugated single-chain antibodies. Wen et al., (2001) (Bioconj. Chem. 12:545-553) disclose conjugating antibodies with PEG which is attached to a radiometal chelator (diethylenetriaminpentaacetic acid (DTPA)).

The antibodies and antibody fragments or the MDL-1 soluble proteins or fragments thereof of the invention may also be conjugated with labels such as ⁹⁹Tc, ⁹⁰Y, ¹¹¹In, ³²P, ¹⁴C, ¹²⁵I, ³H, ¹³¹I, ¹¹C, ¹⁵O, ¹³N, ¹⁸F, ³⁵S, ⁵¹Cr, ⁵⁷To, ²²⁶Ra, ⁶⁰Co, ⁵⁹Fe, ⁵⁷Se, ¹⁵²Eu, ⁶⁷CU, ²¹⁷Ci, ²¹¹At, ²¹²Pb, ⁴⁷Sc, ¹⁰⁹Pd, ²³⁴Th, and ⁴⁰K, ¹⁵⁷Gd, ⁵⁵Mn, ⁵²Tr and ⁵⁶Fe.

The antibodies and antibody fragments or the MDL-1 soluble proteins or fragments thereof of the invention may also be conjugated with fluorescent or chemilluminescent labels, including fluorophores such as rare earth chelates, fluorescein and its derivatives, rhodamine and its derivatives, isothiocyanate, phycoerythrin, phycocyanin, allophycocyanin, o-phthaladehyde, fluorescamine, ¹⁵²Eu, dansyl, umbelliferone, luciferin, luminal label, isoluminal label, an aromatic acridinium ester label, an imidazole label, an acridimium salt label, an oxalate ester label, an aequorin label, 2,3-dihydrophthalazinediones, biotin/avidin, spin labels and stable free radicals.

The antibody molecules or soluble MDL-1 proteins may also be conjugated to a cytotoxic factor such as diptheria toxin, Pseudomonas aeruginosa exotoxin A chain, ricin A chain, abrin A chain, modeccin A chain, alpha-sarcin, Aleurites fordii proteins and compounds (e.g., fatty acids), dianthin proteins, Phytoiacca americana proteins PAPI, PAPII, and PAP-S, momordica charantia inhibitor, curcin, crotin, saponaria officinalis inhibitor, mitogellin, restrictocin, phenomycin, and enomycin.

Any method known in the art for conjugating the antibody molecules or protein molecules of the invention to the various moieties may be employed, including those methods described by Hunter et al., (1962) Nature 144:945; David et al., (1974) Biochemistry 13:1014; Pain etal., (1981) J. Immunol. Meth. 40:219; and Nygren, J., (1982) Histochem. and Cytochem. 30:407. Methods for conjugating antibodies and proteins are conventional and very well known in the art.

Antigenic (i.e., immunogenic) fragments of the MDL-1 peptides of the invention are within the scope of the present invention. Antigenic fragments may be joined to other materials, such as fused or covalently joined polypeptides, to be used as immunogens. The antigenic peptides may be useful for preparing antibody molecules which recognize MDL-1 or any fragment thereof. An antigen and its fragments may be fused or covalently linked to a variety of immunogens, such as keyhole limpet hemocyanin, bovine serum albumin, or ovalbumin (Coligan et al. (1994) Current Protocols in Immunol., Vol. 2, 9.3-9.4, John Wiley and Sons, New York, N.Y.). Peptides of suitable antigenicity may be selected from the polypeptide target, using an algorithm, see, e.g., Parker et al. (1986) Biochemistry 25:5425-5432; Jameson and Wolf (1988) Cabios 4:181-186; Hopp and Woods (1983) Mol. Immunol. 20:483-489.

Although it is not always necessary, when MDL-1 peptides are used as antigens to elicit antibody production in an immunologically competent host, smaller antigenic fragments are preferably first rendered more immunogenic by cross-linking or concatenation, or by coupling to an immunogenic carrier molecule (i.e., a macromolecule having the property of independently eliciting an immunological response in a host animal, such as diptheria toxin or tetanus). Cross-linking or conjugation to a carrier molecule may be required because small polypeptide fragments sometimes act as haptens (molecules which are capable of specifically binding to an antibody but incapable of eliciting antibody production, i.e., they are not immunogenic). Conjugation of such fragments to an immunogenic carrier molecule renders them more immunogenic through what is commonly known as the “carrier effect”.

Carrier molecules include, e.g., proteins and natural or synthetic polymeric compounds such as polypeptides, polysaccharides, lipopolysaccharides, etc. Protein carrier molecules are especially preferred, including, but not limited to, keyhole limpet hemocyanin and mammalian serum proteins such as human or bovine gammaglobulin, human, bovine or rabbit serum albumin, or methylated or other derivatives of such proteins. Other protein carriers will be apparent to those skilled in the art. Preferably, the protein carrier will be foreign to the host animal in which antibodies against the fragments are to be elicited.

Covalent coupling to the carrier molecule may be achieved using methods well known in the art; the exact choice of which will be dictated by the nature of the carrier molecule used. When the immunogenic carrier molecule is a protein, the fragments of the invention may be coupled, e.g., using water-soluble carbodiimides such as dicyclohexylcarbodiimide or glutaraldehyde.

Coupling agents, such as these, may also be used to cross-link the fragments to themselves without the use of a separate carrier molecule. Such cross-linking into aggregates may also increase immunogenicity. Immunogenicity may also be increased by the use of known adjuvants, alone or in combination with coupling or aggregation.

Adjuvants for the vaccination of animals include, but are not limited to, Adjuvant 65 (containing peanut oil, mannide monooleate and aluminum monostearate); Freund's complete or incomplete adjuvant; mineral gels such as aluminum hydroxide, aluminum phosphate and alum; surfactants such as hexadecylamine, octadecylamine, lysolecithin, dimethyldioctadecylammonium bromide, N,N-dioctadecyl-N′,N′-bis(2-hydroxymethyl) propanediamine, methoxyhexadecylglycerol and pluronic polyols; polyanions such as pyran, dextran sulfate, poly IC, polyacrylic acid and carbopol; peptides such as muramyl dipeptide, dimethylglycine and tuftsin; and oil emulsions. The polypeptides could also be administered following incorporation into liposomes or other microcarriers.

Information concerning adjuvants and various aspects of immunoassays are disclosed, e.g., in the series by P. Tijssen, Practice and Theory of Enzyme Immunoassays, 3rd Edition, 1987, Elsevier, New York. Other useful references covering methods for preparing polyclonal antisera include Microbiology, 1969, Hoeber Medical Division, Harper and Row; Landsteiner, Specificity of Serological Reactions, 1962, Dover Publications, New York, and Williams, et al., Methods in Immunology and Immunochemistry, Vol. 1, 1967, Academic Press, New York.

The anti-MDL-1 “antibody molecules” of the invention include, but are by no means not limited to, anti-MDL-1 antibodies (e.g., monoclonal antibodies, polyclonal antibodies, bispecific antibodies and anti-idiotypic antibodies) and fragments, preferably antigen-binding fragments, thereof, such as Fab antibody fragments, F(ab)₂ antibody fragments, Fv antibody fragments (e.g., V_{H} or V_{L}), single chain Fv antibody fragments and dsFv antibody fragments. Furthermore, the antibody molecules of the invention may be fully human antibodies, mouse antibodies, rabbit antibodies, chicken antibodies, human/mouse chimeric antibodies or humanized antibodies.

The anti-MDL-1 antibody molecules of the invention preferably recognize human or mouse MDL-1 peptides of the invention; however, the present invention includes antibody molecules which recognize MDL-1 peptides from different species, preferably mammals (e.g., pig, rat, rabbit, sheep or dog).

The present invention also includes complexes comprising the MDL-1 peptides of the invention and one or more antibody molecules. Such complexes may be made by simply contacting the antibody molecule with its cognate peptide.

Various methods may be used to make the antibody molecules of the invention. In preferred embodiments, the antibodies of the invention are produced by methods which are similar to those disclosed in U.S. Pat. Nos. 5,625,126; 5,877,397; 6,255,458; 6,023,010 and 5,874,299. Hybridoma cells which produce monoclonal, fully human anti-MDL-1 peptide antibodies may then be produced by methods which are commonly known in the art. These methods include, but are not limited to, the hybridoma technique originally developed by Kohler et al., (1975) (Nature 256:495-497), as well as the trioma technique (Hering et al., (1988) Biomed. Biochim. Acta. 47:211-216 and Hagiwara et al., (1993) Hum. Antibod. Hybridomas 4:15), the human B-cell hybridoma technique (Kozbor et al., (1983) Immunology Today 4:72 and Cote et al., (1983) Proc. Natl. Acad. Sci. U.S.A 80:2026-2030), and the EBV-hybridoma technique (Cole et al., in Monoclonal Antibodies and Cancer Therapy, Alan R. Liss, Inc., pp. 77-96, 1985). Again, ELISA may be used to determine if hybridoma cells are expressing anti-MDL-1 peptide antibodies.

Purification of antigen is not necessary for the generation of antibodies. Immunization may be performed by DNA vector immunization, see, e.g., Wang, et al. (1997) Virology 228:278-284. Alternatively, animals may be immunized with cells bearing the antigen of interest. Splenocytes may then be isolated from the immunized animals, and the splenocytes may be fused with a myeloma cell line to produce a hybridoma (Meyaard et al. (1997) Immunity 7:283-290; Wright et al. (2000) Immunity 13:233-242; Preston et al. (1997) Eur. J. Immunol. 27:1911-1918). Resultant hybridomas may be screened for production of the desired antibody by functional assays or biological assays, that is, assays not dependent on possession of the purified antigen. Immunization with cells may prove superior for antibody generation than immunization with purified antigen (Kaithamana et al. (1999) J. Immunol. 163:5157-5164).

Antibody to antigen and ligand to receptor binding properties may be measured, e.g., by surface plasmon resonance (Karlsson et al. (1991) J. Immunol. Methods 145:229-240; Neri et al. (1997) Nat. Biotechnol. 15:1271-1275; Jonsson et al. (1991) Biotechniques 11:620-627) or by competition ELISA (Friguet et al. (1985) J. Immunol. Methods 77:305-319; Hubble (1997) Immunol. Today 18:305-306). Antibodies maybe used for affinity purification to isolate the antibody's target antigen and associated bound proteins, see, e.g., Wilchek et al. (1984) Meth. Enzymol. 104:3-55.

Antibodies that specifically bind to variants of MDL-1, where the variant has substantially the same nucleic acid and amino acid sequence as those recited herein, but possessing substitutions that do not substantially affect the functional aspects of the nucleic acid or amino acid sequence, are within the definition of the contemplated methods. Variants with truncations, deletions, additions, and substitutions of regions which do not substantially change the biological functions of these nucleic acids and polypeptides are within the definition of the contemplated methods.

### Antibody Binding Assays

The antibodies of the invention may be assayed for immunospecific binding by any method known in the art. The immunoassays which may be used include, but are not limited to, competitive and non-competitive assay systems using techniques, such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art (see, e.g., Ausubel et al, eds, 1994, Current Protocols in Molecular Biology, Vol. 1, John Wiley & Sons, Inc., New York, which is incorporated by reference herein in its entirety). Exemplary immunoassays are described briefly below (but are not intended by way of limitation).

Immunoprecipitation protocols generally comprise lysing a population of cells in a lysis buffer, such as RIPA buffer (1% NP-40 or Triton X-100, 1% sodium deoxycholate, 0.1% SDS, 0.15 M NaCl, 0.01 M sodium phosphate at pH 7.2, 1% Trasylol) supplemented with protein phosphatase and/or protease inhibitors (e.g., EDTA, PMSF, aprotinin, sodium vanadate), adding the antibody of interest to the cell lysate, incubating for a period of time (e.g., 1-4 hours) at 4° C., adding protein A and/or protein G sepharose beads to the cell lysate, incubating for about an hour or more at 4° C., washing the beads in lysis buffer and resuspending the beads in SDS/sample buffer. The ability of the antibody of interest to immunoprecipitate a particular antigen may be assessed by, e.g., western blot analysis. One of skill in the art would be knowledgeable as to the parameters that may be modified to increase the binding of the antibody to an antigen and decrease the background (e.g., pre-clearing the cell lysate with sepharose beads). For further discussion regarding immunoprecipitation protocols see, e.g., Ausubel et al, eds, 1994, Current Protocols in Molecular Biology, Vol. 1, John Wiley & Sons, Inc., New York at 10.16.1.

Western blot analysis generally comprises preparing protein samples, electrophoresis of the protein samples in a polyacrylamide gel (e.g., 8%-20% SDS-PAGE depending on the molecular weight of the antigen), transferring the protein sample from the polyacrylamide gel to a membrane such as nitrocellulose, PVDF or nylon, blocking the membrane in blocking solution (e.g., PBS with 3% BSA or non-fat milk), washing the membrane in washing buffer (e.g., PBS-Tween 20), blocking the membrane with primary antibody (the antibody of interest) diluted in blocking buffer, washing the membrane in washing buffer, blocking the membrane with a secondary antibody (which recognizes the primary antibody, e.g., an anti-human antibody) conjugated to an enzymatic substrate (e.g., horseradish peroxidase or alkaline phosphatase) or radioactive molecule (e.g., 32P or 125I) diluted in blocking buffer, washing the membrane in wash buffer, and detecting the presence of the antigen. One of skill in the art would be knowledgeable as to the parameters that may be modified to increase the signal detected and to reduce the background noise. For further discussion regarding western blot protocols see, e.g., Ausubel et al, eds, 1994, Current Protocols in Molecular Biology, Vol. 1, John Wiley & Sons, Inc., New York at 10.8.1.

ELISAs comprise preparing antigen, coating the well of a 96 well microtiter plate with the antigen, adding the antibody of interest conjugated to a detectable compound such as an enzymatic substrate (e.g., horseradish peroxidase or alkaline phosphatase) to the well and incubating for a period of time, and detecting the presence of the antigen. In ELISAs the antibody of interest does not have to be conjugated to a detectable compound; instead, a second antibody (which recognizes the antibody of interest) conjugated to a detectable compound may be added to the well. Further, instead of coating the well with the antigen, the antibody may be coated to the well. In this case, a second antibody conjugated to a detectable compound may be added following the addition of the antigen of interest to the coated well. One of skill in the art would be knowledgeable as to the parameters that may be modified to increase the signal detected as well as other variations of ELISAs known in the art. For further discussion regarding ELISAs see, e.g., Ausubel et al, eds, 1994, Current Protocols in Molecular Biology, Vol. 1, John Wiley & Sons, Inc., New York at 11.2.1.

The binding affinity of an antibody to an antigen and the off-rate of an antibody-antigen interaction may be determined by competitive binding assays. One example of a competitive binding assay is a radioimmunoassay comprising the incubation of labeled antigen (e.g., 3H or 125I) with the antibody of interest in the presence of increasing amounts of unlabeled antigen, and the detection of the antibody bound to the labeled antigen. The affinity of the antibody of interest for a particular antigen and the binding off-rates may be determined from the data by scatchard plot analysis. Competition with a second antibody may also be determined using radioimmunoassays. In this case, the antigen is incubated with antibody of interest conjugated to a labeled compound (e.g., 3H or 125I) in the presence of increasing amounts of an unlabeled second antibody.

The ability of an antibody to preferentially and specifically bind one antigen compared to another antigen may be determined using any method known in the art. By way of non-limiting example, an antibody may be considered to bind a first antigen preferentially if it binds said first antigen with a dissociation constant (K_{D}) that is less than the antibody's K_{D} for the second antigen. In another non-limiting embodiment, an antibody may be considered to bind a first antigen preferentially if it binds said first antigen with an affinity (i.e., K_{D}) that is at least one order of magnitude less than the antibody's K_{D} for the second antigen. In another non-limiting embodiment, an antibody may be considered to bind a first antigen preferentially if it binds said first antigen with an affinity (i.e., K_{D}) that is at least two orders of magnitude less than the antibody's K_{D} for the second antigen.

In another non-limiting embodiment, an antibody may be considered to bind a first antigen preferentially if it binds said first antigen with an off rate (K_{off}) that is less than the antibody's K_{off} for the second antigen. In another non-limiting embodiment, an antibody may be considered to bind a first antigen preferentially if it binds said first antigen with a K_{off} that is at least one order of magnitude less than the antibody's K_{off} for the second antigen. In another non-limiting embodiment, an antibody may be considered to bind a first antigen preferentially if it binds said first antigen with a K_{off} that is at least two orders of magnitude less than the antibody's K_{off} for the second antigen.

Antibodies of the present invention may also be described or specified in terms of their cross-reactivity. Antibodies that do not bind any other analog, ortholog, or homolog of a polypeptide of the present invention are included. Antibodies that bind polypeptides with at least 100%, at least 99%, at least 98%, at least 97%, at least 96%, at least 95%, at least 94%, at least 93%, at least 92%, at least 91%, at least 90%, at least 80%, at least 70%, at least 60%, and at least 50% identity (as calculated using methods known in the art and described herein) to a polypeptide of the present invention are also included in the present invention.

Antibodies that do not bind polypeptides with less than 100%, less than 99%, less than 98%, less than 97%, less than 96%, less than 95%, less than 94%, less than 93%, less than 92%, less than 91%, less than 90%, less than 80%, less than 70%, less than 60%, and less than 50% identity (as calculated using methods known in the art and described herein) to a polypeptide of the present invention are also included in the present invention. Further included in the present invention are antibodies which bind polypeptides encoded by polynucleotides which hybridize to a polynucleotide of the present invention under stringent hybridization conditions (as described herein). Antibodies of the present invention may also be described or specified in terms of their binding affinity to a polypeptide of the invention.

### Therapeutic and Diagnostic Uses

The invention provides methods for the diagnosis and treatment of proliferative disorders, e.g., cancer. The invention provides methods for the diagnosis and treatment of proliferative disorders, cancer, e.g., tumors. The invention also provides methods for the diagnosis and treatment of solid tumors. The methods may comprise the use of a binding composition specific for a polypeptide or nucleic acid of MDL-1, e.g., an antibody or a antigen binding fragment thereof or a soluble MDL-1 protein or a nucleic acid probe or primer. Control binding compositions are also provided, e.g., control antibodies, see, e.g., Lacey et al. (2003) Arthritis Rheum. 48:103-109; Choy and Panayi (2001) New Engl. J. Med. 344:907-916; Greaves and Weinstein (1995) New Engl. J. Med. 332:581-588; Robert and Kupper (1999) New Engl. J. Med. 341:1817-1828; Lebwohl (2003) Lancet 361:1197-1204.

Methods for co-administration or treatment with a second therapeutic agent, e.g., a cytokine, chemotherapeutic agent, antibiotic, or radiation, are well known in the art (Hardman, et al. (eds.) (2001) Goodman and Gilman's The Pharmacological Basis of Therapeutics, 10th ed., McGraw-Hill, New York, N.Y.; Poole and Peterson (eds.) (2001) Pharmacotherapeutics for Advanced Practice:A Practical Approach, Lippincott, Williams & Wilkins, Phila., Pa.; Chabner and Longo (eds.) (2001) Cancer Chemotherapy and Biotherapy, Lippincott, Williams & Wilkins, Phila., Pa.). An effective amount of therapeutic will decrease the symptoms typically by at least 10%; usually by at least 20%; preferably at least 30%; more preferably at least 40%, and most preferably by at least 50%.

Formulations of therapeutic and diagnostic agents may be prepared for storage by mixing with physiologically acceptable carriers, excipients, or stabilizers in the form of, e.g., lyophilized powders, slurries, aqueous solutions or suspensions, see, e.g., Hardman, et al. (2001) Goodman and Gilman's The Pharmacological Basis of Therapeutics, McGraw-Hill, New York, N.Y.; Gennaro (2000) Remington: The Science and Practice of Pharmacy, Lippincott, Williams, and Wilkins, New York, N.Y.; Avis, et al. (eds.) (1993) Pharmaceutical Dosage Forms: Parenteral Medications, Marcel Dekker, N.Y.; Lieberman, et al. (eds.) (1990) Pharmaceutical Dosage Forms: Tablets, Marcel Dekker, N.Y.; Lieberman, et al. (eds.) (1990) Pharmaceutical Dosage Forms: Disperse Systems, Marcel Dekker, N.Y.; Weiner and Kotkoskie (2000) Excipient Toxicity and Safety, Marcel Dekker, Inc., New York, N.Y.;

Determination of the appropriate dose is made by the clinician, e.g., using parameters or factors known or suspected in the art to affect treatment or predicted to affect treatment. Generally, the dose begins with an amount somewhat less than the optimum dose and it is increased by small increments thereafter until the desired or optimum effect is achieved relative to any negative side effects. Important diagnostic measures include those of symptoms of, e.g., the inflammation or level of inflammatory cytokines produced. Preferably, a biologic that will be used is derived from the same species as the animal targeted for treatment, thereby minimizing a humoral response to the reagent.

An effective amount for a particular patient may vary depending on factors such as the condition being treated, the overall health of the patient, the method route and dose of administration and the severity of side affects. When in combination, an effective amount is in ratio to a combination of components and the effect is not limited to individual components alone. Guidance for methods of treatment and diagnosis is available (Maynard, et al. (1996) A Handbook of SOPs for Good Clinical Practice, Interpharm Press, Boca Raton, Fla.; Dent (2001) Good Laboratory and Good Clinical Practice, Urch Publ., London, UK).

The invention also provides a kit comprising a cell and a compartment, a kit comprising a cell and a reagent, a kit comprising a cell and instructions for use or disposal, as well as a kit comprising a cell, compartment, and a reagent.

### Pharmaceutical Compositions

The antibody molecules or soluble MDL-1 proteins of the invention may be administered, preferably for therapeutic purposes, to a subject, preferably in a pharmaceutical composition. Preferably, a pharmaceutical composition includes a pharmaceutically acceptable carrier. The antibody molecules may be used therapeutically (e.g., in a pharmaceutical composition) to target the MDL-1 receptor and, thereby, to treat any medical condition caused or mediated by the receptor. The soluble MDL-1 proteins may be used therapeutically (e.g., in a pharmaceutical composition) to target the MDL-1 receptor ligand and, thereby, to treat any medical condition caused or mediated by the receptor.

Pharmaceutically acceptable carriers are conventional and very well known in the art. Examples include aqueous and nonaqueous carriers, stabilizers, antioxidants, solvents, dispersion media, coatings, antimicrobial agents, buffers, serum proteins, isotonic and absorption delaying agents, and the like that are physiologically compatible. Preferably, the carrier is suitable for injection into a subject's body. Generally, compositions useful for parenteral administration of such drugs are well known; e.g., Remington's Pharmaceutical Science, 17th Ed. (Mack Publishing Company, Easton, Pa., 1990).

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity may be maintained, for example, by the use of coating materials, such as lecithin, by the maintenance of the required particle size in the case of dispersions, and by the use of surfactants.

The pharmaceutical compositions of the invention may be administered in conjunction with a second pharmaceutical composition or substance. When a combination therapy is used, both compositions may be formulated into a single composition for simultaneous delivery or formulated separately into two or more compositions (e.g., a kit).

Analgesics may include aspirin, acetominophen, codein, morphine, aponorphine, normorphine, etorphine, buprenorphine, hydrocodone, racemorphan, levorphanol, butorphand, methadone, demerol, ibuprofen or oxycodone.

Pharmaceutical compositions of the invention may also include other types of substances, including small organic molecules and inhibitory ligand analogs, which may be identified using the assays described herein.

The formulations may conveniently be presented in unit dosage form and may be prepared by any methods well known in the art of pharmacy. See, e.g., Gilman et al. (eds.) (1990), The Pharmacological Bases of Therapeutics, 8th Ed., Pergamon Press; and Remington's Pharmaceutical Sciences, supra, Easton, Pa.; Avis et al. (eds.) (1993) Pharmaceutical Dosage Forms: Parenteral Medications Dekker, New York; Lieberman et al. (eds.) (1990) Pharmaceutical Dosage Forms: Tablets Dekker, New York; and Lieberman et al. (eds.) (1990), Pharmaceutical Dosage Forms: Disperse Systems Dekker, New York.

The dosage regimen involved in a therapeutic application may be determined by a physician, considering various factors which may modify the action of the therapeutic substance, e.g., the condition, body weight, sex and diet of the patient, the severity of any infection, time of administration, and other clinical factors.

Often, treatment dosages are titrated upward from a low level to optimize safety and efficacy. Dosages may be adjusted to account for the smaller molecular sizes and possibly decreased half-lives (clearance times) following administration.

Typical protocols for the therapeutic administration of such substances are well known in the art. Pharmaceutical compositions of the invention may be administered, for example, by parenteral routes (e.g., intravenous injection, intramuscular injection, subcutaneous injection, intratumoral injection or by infusion) or by a non-parenteral route (e.g., oral administration, pulmonary administration or topical administration).

Compositions may be administered with medical devices known in the art. For example, in a preferred embodiment, a pharmaceutical composition of the invention may be administered by injection with a hypodermic needle.

The pharmaceutical compositions of the invention may also be administered with a needleless hypodermic injection device; such as the devices disclosed in U.S. Pat. Nos. 5,399,163; 5,383,851; 5,312,335; 5,064,413; 4,941,880; 4,790,824 or 4,596,556.

Examples of well-known implants and modules useful in the present invention include: U.S. Pat. No. 4,487,603, which discloses an implantable micro-infusion pump for dispensing medication at a controlled rate; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments.

### Anti-Sense Molecules

The present invention also encompasses anti-sense oligonucleotides capable of specifically hybridizing to nucleic acids (e.g., genomic DNA or mRNA) encoding MDL-1 peptides of the invention, preferably having an amino acid sequence defined by any of SEQ ID NOs: 2 or 4 or a subsequence thereof so as to prevent expression of the nucleic acid.

This invention further provides pharmaceutical compositions comprising (a) an amount of an oligonucleotide effective to modulate the activity of the MDL-1 receptor by passing through a cell membrane and binding specifically with mRNA encoding a MDL-1 peptide of the invention in the cell so as to prevent its translation and (b) a pharmaceutically acceptable carrier capable of passing through a cell membrane. In an embodiment, the oligonucleotide is coupled to a substance that inactivates mRNA (e.g., a ribozyme).

### EXAMPLES

The following Examples exemplify the present invention and should not be construed to limit the broad scope of the invention.

I. mRNA Expression of MDL-1.

MDL-1 (a.k.a. CLECSF5, C-type Lectin Superfamily 5; see, e.g., Ebner et al. (2003) Proteins 53:44-55; and Drickamer (1999) Curr. Opin. Struct. Biol. 9:585-590) expression levels in various human tumor samples were determined. Tumor tissues were collected for each case, and when possible matching normal adjacent tissue was also collected. All tissues were screened in-house by pathologists to confirm staging diagnoses. Total RNA was prepared from tissue by standard methodologies and reverse transcribed. Real-time quantitative PCR was performed by standard methodologies. The absence of genomic DNA contamination was confirmed using primers that recognize genomic region of the CD4 promoter. Ubiquitin levels were measured in a separate reaction and used to normalize the data by the Δ-Δ Ct method. See, e.g., User Bulletin #2 (1997) Applied Biosystems, Foster City, Calif. (Using the mean cycle threshold value for ubiquitin and MDL-1 for each sample, the equation 1.8 e (Ct ubiquitin minus Ct MDL-1)×10⁴ was used to obtain the normalized values.) Kruskal-Wallis non-parametric statistical analysis was performed on log transformed data (median method). See, e.g., Hollander and Wolfe (1973) Nonparametric Statistical Interference, John Wiley and Sons, New York, N.Y., pp. 115-120. These methods were used in the cancer panels below.

IA. Human Melanoma Panel.

The melanoma panel included 15 control normal skin samples; 86 normal adjacent tissues, matched with tumor cases; and 87 melanoma cases, ordered by stage on panel. Total RNA was prepared from tissue by standard methodologies and reverse transcribed. Real-time quantitative PCR was performed by standard methodologies.

The control tissues (n=15) had a median MDL-1 expression value of 3.66, while melanoma samples of stage I, stage II SS, stage II NM, stage II melanoma (general) and stage III/IV had median MDL-1 expression values of 18.83 (5.1 fold), 8.79 (2.4 fold), 9.75 (2.6 fold), 10.81 (2.9 fold), and 11.66 (3.1 fold), respectively (Table 2). Kruskal-Wallis median analysis on log-transformed data showed statistically significant elevation in stage I (P<0.001), stage II nodular (P<0.01) and stage III/IV (P<0.01) melanoma samples compared to normal samples.

TABLE 2
Human melanoma panel: Expression of MDL-1 by quantitative real-
time PCR analysis, relative to ubiquitin and log transformed.

| | Median MDL-1 expression | Fold increase over |
|---|---|---|
| Type of tissue | value | control |
| Control tissue | 3.66 | |
| Stage I melanoma | 18.83 | 5.1 |
| Stage II SS | 8.79 | 2.4 |
| Stage II NM | 9.75 | 2.6 |
| Stage II melanoma | 10.81 | 2.9 |
| (general) | | |
| Stage II/IV melanoma | 11.66 | 3.1 |

IB. Human Ovarian Tumor Panel.

The ovarian panel included 20 control ovary tissues; 35 normal adjacent tissues, matched with tumor cases; and 36 papillary serous cystadenocarcinoma tissues, ordered by stage/differentiation on panel. Total RNA was prepared from tissue by standard methodologies and reverse transcribed. Real-time quantitative PCR was performed by standard methodologies.

The control tissues (n=20) had a median MDL-1 expression value of 2.95, while the ovarian tumor samples of stage I, stage II, and stage III/IV had median MDL-1 expression values of 46.37 (15.7 fold), 40.55 (13.7 fold), and 13.35 (4.5 fold), respectively (Table 3). Kruskal-Wallis median analysis on log-transformed data showed statistically significant elevation in all three stage groups, with P values of less than 0.001 for all, compared to normal control tissues.

TABLE 3
Human ovarian cancer panel: Expression of MDL-1 by quantitative
real-time PCR analysis, relative to ubiquitin and log transformed.

| | Median | |
|---|---|---|
| | MDL-1 expression | Fold increase over |
| Type of tissue | value | control |
| Control tissue | 2.95 | |
| Stage 1 ovarian tumor | 46.37 | 15.7 |
| Stage II ovarian tumor | 40.55 | 13.7 |
| Stage III/IV ovarian tumor | 13.35 | 4.5 |

IC. Human Breast Tumor Panel.

The breast tumor panel included 18 control breast tissues; 79 normal adjacent tissues, matched with tumor cases (where available); and 91 breast tumor cases: 6 ductal carcinoma in situ, 64 infiltrating ductal carcinoma (IDC), 4 mucinous IDC, 2 mixed IDC, 13 infiltrating lobular carcinoma and 2 medullary carcinoma tissues, ordered by stage/differentiation on panel. Total RNA was prepared from tissue by standard methodologies and reverse transcribed. Real-time quantitative PCR was performed by standard methodologies.

The control tissues (n=18) had a median MDL-1 expression value of 1.03, while the breast IDC tumor samples of stage I, stage II, and stage III/IV and lobular (all stages) had median MDL-1 expression values of 7.37 (7.1 fold), 7.12 (6.9 fold), 10.39 (10 fold), and 3.71 (3.6 fold), respectively (Table 4). Data from groups with small ‘n’ were excluded from statistical analysis (mucinous IDC, mixed IDC and medullary carcinomas). Kruskal-Wallis median analysis on log-transformed data showed statistically significant elevation in all three stage groups of IDC, with P values of less than 0.001 for all, and significance in lobular carcinoma (P<0.01) compared to normal control plus normal adjacent tissues (n=97).

TABLE 4
Human Breast Cancer Panel: Expression of MDL-1 by quantitative
real-time PCR analysis, relative to ubiquitin and log transformed.

| | Median | |
|---|---|---|
| | MDL-1 expression | Fold increase over |
| Type of tissue | value | control |
| Control tissue | 1.03 | |
| Stage I breast IDC tumor | 7.37 | 7.1 |
| Stage II breast IDC tumor | 7.12 | 6.9 |
| Stage III/IV breast IDC | 10.39 | 10.0 |
| tumor | | |
| Lobular breast tumor (all | 3.71 | 3.6 |
| stages) | | |

ID. Human Colorectal Tumor Panel.

The colorectal tumor panel included 11 control colon tissues; 40 normal adjacent tissues, matched with tumor cases; and 40 colorectal adenocarcinoma tissues, ordered by stage/differentiation on panel. Total RNA was prepared from tissue by standard methodologies and reverse transcribed. Real-time quantitative PCR was performed by standard methodologies.

The control tissues (n=11) had a median MDL-1 expression value of 1.75, while the colorectal tumor samples of stage I, stage II, and stage III/IV had median MDL-1 expression values of 8.05 (4.6 fold), 36.59 (20.9 fold), and 14.16 (8 fold), respectively (Table 5). Kruskal-Wallis median analysis on log-transformed data showed statistically significant elevation in stage II and stage III/IV groups, with P values of less than 0.001 for both, compared to normal control plus normal adjacent tissues (n=51).

TABLE 5
Human Colorectal Cancer Panel: Expression of MDL-1 by
quantitative real-time PCR analysis, relative to ubiquitin
and log transformed.

| | Median MDL-1 | Fold increase over |
|---|---|---|
| Type of tissue | expression value | control |
| Control tissue | 1.75 | |
| Stage 1 colorectal tumor | 8.05 | 4.6 |
| Stage II colorectal tumor | 36.59 | 20.9 |
| Stage III/IV colorectal tumor | 14.16 | 8.0 |

IE. Human Renal Tumor Panel.

The renal tumor panel included 12 control kidney tissues; 30 normal adjacent tissues, matched with tumor cases; and 31 clear cell renal carcinomas tissues, ordered by stage/differentiation on panel. Total RNA was prepared from tissue by standard methodologies and reverse transcribed. Real-time quantitative PCR was performed by standard methodologies.

The control tissues (n=12) had a median MDL-1 expression value of 1.73, while the renal tumor samples of stage I/II and stage III/IV had median MDL-1 expression values of 5.09 (2.9 fold) and 9.35 (5.4 fold), respectively (Table 6). Samples from stages I and II and from stages III and IV were grouped due to low ‘n’ of samples. Kruskal-Wallis median analysis on log-transformed data showed statistically significant elevation in both stage I/II (P<0.01) and stage III/IV groups (P<0.001) compared to normal control plus normal adjacent tissues (n=42).

TABLE 6
Human Renal Cancer Panel: Expression of MDL-1 by quantitative
real-time PCR analysis, relative to ubiquitin and log transformed.

| | Median | |
|---|---|---|
| | MDL-1 expression | Fold increase over |
| Type of tissue | value | control |
| Control tissue | 1.73 | |
| Stage I/II renal tumor | 5.09 | 2.9 |
| Stage III/IV renal tumor | 9.35 | 5.4 |

IF. Human Stomach Tumor Panel.

The stomach tumor panel included 12 control stomach tissues; 64 normal adjacent tissues, matched with tumor cases (where available); and 75 stomach adenocarcinoma tissues, ordered by stage/differentiation on panel. Total RNA was prepared from tissue by standard methodologies and reverse transcribed. Real-time quantitative PCR was performed by standard methodologies.

The control tissues (n=12) had a median MDL-1 expression value of 0.39, while the stomach tumor samples of stage I, stage II, stage IIIA, stage IIIB and stage IV had median MDL-1 expression values of 4.42 (11.3 fold), 4.14 (10.6 fold), 9.77 (25 fold), 8.98 (23 fold) and 7.03 (18 fold), respectively (Table 7). Kruskal-Wallis median analysis on log-transformed data showed statistically significant elevation in stage II (P<0.05), stage IIIA and IIIB (P<0.001 for both) and stage IV (P<0.01) groups, compared to normal control plus normal adjacent tissues (n=76) (see attached graph).

TABLE 7
Human Stomach Cancer Panel: Expression of MDL-1 by
quantitative real-time PCR analysis, relative to ubiquitin.

| | Median | |
|---|---|---|
| | MDL-1 expression | Fold increase over the |
| Type of tissue | value | control |
| Control tissue | 0.39 | |
| Stage I stomach tumor | 4.42 | 11.3 |
| Stage II stomach tumor | 4.14 | 10.6 |
| Stage III A stomach tumor | 9.77 | 25.0 |
| Stage III B stomach tumor | 8.98 | 23.0 |
| Stage IV stomach tumor | 7.03 | 18.0 |

### CONCLUSION

The expression level (value normalized to ubiquitin and log transformed) corresponded to the amount of MDL-1 expressed in the tissue sample, such that the higher the expression level, the greater the amount of MDL-1 expressed in the tissue sample. The above experimental results demonstrate that MDL-1 expression is significantly elevated in melanoma, ovarian, breast, colorectal, renal and stomach cancers relative to controls.

II. Antibody Generation

Generation of antibodies that specifically bind human and mouse MDL-1.

Antibodies that specifically bind human MDL-1 (huMDL-1) or mouse MDL-1 (muMDL-1) were generated by immunizing rats with an immunogenic fusion protein comprising of the extracellular domain of mouse MDL-1 (amino acid residues 26-190 of SEQ ID NO:4) fused to the Fc domain of human immunoglobulin (MuMDL-1-huIg). Immunizations were continued for several months, at which time the animals were sacrificed and their spleen cells were fused by standard hybridomas protocols to mouse myeloma cells. Anti-human MDL-1 monoclonal antibodies were generated by immunizing Balb/C mice with an immunogenic fusion protein comprising of the extracellular domain of huMDL-1 (amino acid residues 26 to 188 of SEQ ID NO:2) fused to the Fc domain of human immunoglobulin (huMDL-1-huIg). Similar procedures for generation of hybridomas noted above were used.

IIB. Screening for Antibodies that Specifically Bind to MDL-1.

Antibodies that specifically bind muMDL-1 were screened using the supernatants from fused hybrids and subjecting the supernatants to differential ELISA techniques. The anti-muMDL-1 monoclonal antibodies were further tested for specificity by FACS analysis of muMDL-1 cell lines and immunoprecipitation of MDL-1/DAP12 complex from cells expressing muMDL-1. Monoclonal antibodies specific for human MDL-1 were generated from immunized spleen/myeloma fusion hybrids and verified for specificity by FACS and immunoprecipitation of MDL-1 expressed by cell lines.

III. Immunohistochemistry (IHC)

Human tumor biopsies and normal adjacent tissues were obtained from patients undergoing tumor resection surgery and immediately snap frozen in liquid nitrogen. Frozen tissue fragments (1-3 mm) were embedded in OCT and additionally frozen by liquid nitrogen flotation. All frozen tissues were then stored at −80° C. Cryostat sections (5-8 um) were fixed in cold 80% acetone and 20% methanol, air dried, then blocked with 15% normal goat serum for 30 minutes at room temperature. Sections were then incubated in primary antibodies (3 μg/ml) for 2 hours at room temperature, extensively washed in PBS, and further incubated 1 hour in Biotin-conjugated goat anti-rat IgG or Biotin conjugated goat anti-mouse IgG (Vector Lab, Burlingame, Calif.). Sections were then incubated in Vectastain ABC reagent for 30 minutes (Vector Labs, Burlingame, Calif.), washed three times in PBS and then incubated in peroxidase substrate for 5-10 minutes. Sections were counter stained with hematoxylin, permanently mounted and examined under a Nikon E800 microscope. Immunostaining can also be performed on paraffin-embedded tissues.

Consistent with the mRNA expression analysis, MDL-1 was strongly expressed on the majority of infiltrating leukocytes in melanoma, ovarian adenocarcinomas, breast invasive ductal carcinomas, colorectal adenocarcinomas, stomach adenocarcinomas and renal clear cell carcinomas. MDL-1 was not expressed by the tumor cells in these carcinomas, but was exclusively expressed by the large number of tumor-infiltrating leukocytes, primarily of the myeloid/macrophage lineage.

IV. Phenotyping of Infiltrating MDL-1 Positive Leukocytes

MDL-1 positive leukocytes may be obtained from human tumor biopsies. Phenotyping of the leukocytes may be performed as described by Mantovani et al. ((2002) TRENDS in Immunol. 23:549-555). Two color FACS analysis using markers for polarized M1 and polarized M2 macrophages may reveal that the MDL-1 positive leukocytes are of the M2 phenotype. The majority of MDL-1 positive leukocytes may co-express the macrophage/monocyte markers, CD68, CD11b, and CD206.

The present invention should not be limited in scope by the specific embodiments described herein. Indeed, various modifications of the invention, in addition to those described herein, will become apparent to those skilled in the art from the foregoing description. Such modifications fall within the scope of the appended claims.

Patents, patent applications, publications, product descriptions and protocols are cited throughout this application, the disclosures of which are incorporated herein by reference in their entireties.

1. A method for diagnosing cancer or detecting a tumor comprising:
(a) measuring levels of myeloid DAP12-associating lectin-1 (MDL-1) expression in a cell or tissue; and
(b) comparing measured levels of MDL-1 with expression levels of MDL-1 in a cell or tissue from a control, wherein an increase in measured levels of MDL-1 expression compared to the control is associated with cancer or the presence of a tumor.

2. The method of claim 1, wherein the MDL-1 is a polypeptide or a nucleic acid.

3. The method of claim 1, wherein the MDL-1 has an amino acid sequence at least 90% identical to SEQ ID NO: 2.

4. The method of claim 1, wherein the MDL-1 comprises an amino acid sequence of SEQ ID NO: 2.

5. The method of claim 1, wherein the MDL-1 is present on tumor-infiltrating leukocytes within a tumor.

6. The method of claim 1, wherein the cancer is selected from the group consisting of melanoma, ovarian cancer, breast cancer, colorectal cancer, renal cancer and stomach cancer.

7. The method of claim 1, wherein the tumor is selected from the group consisting of a solid tumor, a melanoma, an ovarian tumor, a breast tumor, a colorectal tumor, a renal tumor and a stomach tumor.

8. A method for diagnosing or detecting a tumor in a patient comprising:
(a) administering to the patient an antibody or an antigen-binding fragment thereof that binds MDL-1;
(b) measuring a level of binding of the antibody or the antigen-binding fragment thereof in a cell or tissue of the patient; and
(c) comparing the measured level of binding with a level of binding of an antibody or an antigen-binding fragment thereof that binds MDL-1 in a cell or tissue of a control, wherein an increase in measured levels of binding in the patient compared to the control is associated with the presence of the tumor.

9. The method of claim 8, wherein the MDL-1 has an amino acid sequence at least 90% identical to SEQ ID NO: 2.

10. The method of claim 8, wherein the MDL-1 comprises an amino acid sequence of SEQ ID NO: 2.

11. The method of claim 8, wherein the MDL-1 is present on tumor-infiltrating leukocytes within a tumor.

12. The method of claim 8, wherein the tumor is selected from the group consisting of a solid tumor, a melanoma, an ovarian tumor, a breast tumor, a colorectal tumor, a renal tumor and a stomach tumor.

13. The method of claim 8, wherein the antibody or the antigen-binding fragment thereof is selected from the group consisting of a monoclonal antibody, a polyclonal antibody, an activating antibody, an inhibitory antibody, a chimeric antibody, a humanized antibody, a diabody, a single-chain antibody and a fusion protein.

14. The method of claim 8, wherein the antigen-binding fragment is selected from the group consisting of a Fab fragment, a F(ab)₂ fragment and a Fv fragment.

15. The method of claim 8, wherein the antibody or the antigen-binding fragment thereof is bound to a label.

16. The method of claim 15, wherein the label is selected from the group consisting of a radiolabel, a fluorescent label, a chemiluminescent label, a paramagnetic label and an enzymatic label.

17. A method for treating cancer comprising administering to a patient a composition comprising an antibody or an antigen-binding fragment thereof that binds MDL-l, wherein the antibody or the antigen-binding fragment thereof is bound to a cytotoxic agent.

18. The method of claim 17, wherein the MDL-1 has an amino acid sequence at least 90% identical to SEQ ID NO: 2.

19. The method of claim 17, wherein the MDL-1 comprises an amino acid sequence of SEQ ID NO: 2.

20. The method of claim 17, wherein the MDL-1 is present on tumor-infiltrating leukocytes within a tumor.

21. The method of claim 17, wherein the cancer is selected from the group consisting of melanoma, ovarian cancer, breast cancer, colorectal cancer, renal cancer and stomach cancer.

22. The method claim 17, wherein the antibody or the antigen-binding fragment thereof is selected from the group consisting of a monoclonal antibody, a polyclonal antibody, an activating antibody, an inhibitory antibody, a chimeric antibody, a humanized antibody, a diabody, a single-chain antibody and a fusion protein.

23. The method of claim 22, wherein the antigen-binding fragment is selected from the group consisting of a Fab fragment, a F(ab)₂ fragment and a Fv fragment.

24. The method of claim 17, wherein the cytotoxic agent is selected from the group consisting of a drug; a toxin; a compound that emits radiation; a molecule of plant, fungal or bacterial origin; a biological protein; and mixtures thereof.

25. The method of claim 24, wherein the compound that emits radiation is an α-emitter, a β-emitter or a γ-emitter.

26. The method of claim 17, wherein the composition ablates tumor cells, kills tumor cells or reduces tumor size.

27. A method for diagnosing or detecting the presence of a tumor in a patient comprising:
(a) administering to a patient a soluble MDL-1 polypeptide or a fragment thereof;
(b) measuring a level of binding of the polypeptide or the fragment thereof to a ligand in a cell or tissue of the patient; and
(c) comparing the measured level of binding with a level of binding of a soluble MDL-1 polypeptide or a fragment thereof to a ligand in a cell or tissue of a control, wherein an increase in measured levels of binding in the patient compared to the control is associated with the presence of a tumor.

28. The method of claim 27, wherein the soluble MDL-1 polypeptide has an amino acid sequence comprising amino acid residues 26 to 188 of SEQ ID NO: 2.

29. The method of claim 27, wherein the tumor is selected from the group consisting of a solid tumor, a melanoma, an ovarian tumor, a breast tumor, a colorectal tumor, a renal tumor and a stomach tumor.

30. The method of claim 27, wherein the soluble MDL-1 polypeptide or the fragment thereof is bound to a label.

31. The method of claim 30, wherein the label is selected from the group consisting of a radiolabel, a fluorescent label, a chemiluminescent label, a paramagnetic label and an enzymatic label.

32. A method for treating cancer comprising administering to a patient a composition comprising a soluble MDL-1 polypeptide or a fragment thereof that binds to a ligand, wherein the polypeptide or the fragment thereof is bound to a cytotoxic agent.

33. The method of claim 32, wherein the soluble MDL-1 polypeptide has an amino acid sequence comprising amino acid residues 26 to 188 of SEQ ID NO: 2.

34. The method of claim 32, wherein the cancer is selected from the group consisting of melanoma, ovarian cancer, breast cancer, colorectal cancer, renal cancer and stomach cancer.

35. The method of claim 32, wherein the cytotoxic agent is selected from the group consisting of a drug; a toxin; a compound that emits radiation; a molecule of plant, fungal or bacterial origin; a biological protein; and mixtures thereof.

36. The method of claim 35, wherein the compound which emits radiation is an α-emitter, a β-emitter or a γ-emitter.

37. The method of claim 32, wherein the composition ablates tumor cells, kills tumor cells or reduces tumor size.
